(19) United States
(12) Patent Application Publication
(10) Pub. No.: US 2022/0251590 A1
(43) Pub. Date: Aug. 11, 2022

(54) INCREASING WATER USE EFFICIENCY IN PLANTS

(71) Applicant: UNIVERSITY OF ESSEX ENTERPRISES LIMITED, Essex (GB)

(72) Inventors: Tracy Lawson, Essex (GB); Cristobal Uauy, Norfolk (GB); James Simmonds, Norfolk (GB)

(21) Appl. No.: 17/620,625

(22) PCT Filed: Jun. 26, 2020
(86) PCT No.: PCT/GB2020/051550

§ 371 Date: Dec. 17, 2021

(30) Foreign Application Priority Data
Jun. 28, 2019 (GB) 1909299.8

(51) Int. Cl. C12N 15/82

(52) U.S. Cl. CPC C12N 15/8273; C12N 15/8216

(57) ABSTRACT

The invention relates to methods of increasing water use efficiency (WUE) in plants by modulating the stomatal blue light response. The invention further relates to plants having increased WUE, identifying plants with increased WUE and to related methods of increasing drought tolerance and producing food or feed products under water-limited or drought conditions.

### FIELD OF THE INVENTION

The present invention relates to methods of increasing water use efficiency (WUE) in plants by modulating the stomatal blue light response. The invention further relates to plants having increased WUE, identifying plants with increased WUE and to related methods of increasing drought tolerance and producing food or feed products under water-limited or drought conditions.

### BACKGROUND TO THE INVENTION

The global population is predicted to reach nine billion by 2050, increasing demands for food by 70%. Recently, yields for many of the major crops have plateaued and as agricultural land is limited alternative and novel approaches are required to optimise plant performance in a more variable future environment to ensure food sustainability.

Photosynthesis is a key target for increasing yield as it forms the fundamental basis for all plant processes. For photosynthesis to take place, gaseous exchange between the external environment and the plant interior occurs through the stomatal pores on the leaf surface. Stomata are surrounded by two specialised cells, called guard cells that control opening and closing of the pores in response to environmental and internal cues. Stomatal behaviour therefore controls CO₂ uptake for photosynthesis, water loss via transpiration, and as a result leaf temperature through evaporative cooling.

To date the majority of manipulations of photosynthesis have focused on altering enzymatic steps within the Calvin cycle or electron transport chain that are considered as rate limiting or through increasing the CO₂ concentration at the site of carboxylation. However, few if any studies have targeted the manipulation of stomatal behaviour to improve photosynthesis and consequently yield. Guard cells that control the stomatal aperture therefore represent an unexploited but important target for manipulation and improvement of crop productivity.

Stomata open in response to photosynthetically active radiation (PAR), which facilitates CO₂ diffusion into the leaf for photosynthesis, and often results in a close correlation between photosynthetic rates (A) and stomatal conductance (gₛ). However, gₛ is often greater than necessary for maximum photosynthesis under certain conditions resulting in unnecessary water loss with no carbon gain.

For example, guard cells exhibit a specific “blue” light (BL) response that increases stomatal conductance at intensities of blue light too low to drive photosynthesis. This represents an inefficient use of resources as stomata will open more than required to supply sufficient CO₂ to saturate photosynthesis, and therefore the plant loses water without any beneficial effect on CO₂ uptake.

A guard cell includes the signalling components for stomatal cell opening, from blue light perception to cell volume increase. For example, blue light in guard cells is perceived by membrane-associated receptor kinase phototropins (PHOT), PHOT1 and PHOT2. PHOTs are activated by auto-phosphorylation in the presence of blue light. Activated PHOTs each phosphorylate a Ser/Thr protein kinase, Blue Light Signalling 1 (BLUS1) in a light intensity-dependent manner leading to stomatal opening.

There remains a need for methods of increasing WUE in plants. It is an aim of certain embodiments of the present invention to at least partly mitigate the above-mentioned problems associated with the prior art.

### Summary of Certain Embodiments of the Invention

The invention relate to the finding that reducing stomatal sensitivity to blue light optimises resource use in plants, thereby maintaining photosynthetic rates whilst using water more efficiently.

In certain aspects of the invention, decreasing water use in crop species such as wheat enables sustained photosynthetic rates through the grain filling period when water becomes limiting, thus enhancing overall synthetic potential of the plant throughout the cycle and increasing grain yield.

Importantly, reduction of stomatal sensitivity to blue light through the targeting of BLUS1 is restricted to guard cells of the stomata. Advantageously, targeting guard cell specific BLUS1 (vital for blue light response) does not affect the beneficial influence of blue light on mesophyll photosynthesis.

Accordingly, the invention provides:
a method of increasing water use efficiency (WUE) in a plant, the method comprising reducing or abolishing the expression of at least one nucleic acid sequence encoding a Blue Light Signalling 1 (BLUS1) polypeptide and/or reducing or abolishing the activity of a BLUS1 polypeptide in the plant;
a plant or plant part (e.g. seed) obtainable by any method as described herein, typically wherein the plant or plant part is a. crop species such as wheat;
a plant or plant part (e.g. seed) having reduced or abolished expression of at least one nucleic acid encoding a BLUS1 polypeptide and/or reduced or abolished activity of a BLUS1 polypeptide, typically wherein the plant or plant part is a crop species such as wheat;
a method of identifying one or more alleles associated with increased WUE in one or more plants, the method comprising:
(a) detecting in the plant(s) one or more polymorphism(s) in a nucleic acid sequence encoding a BLUS1 polypeptide, wherein the one or more polymorphism(s) are associated with increased WUE; and
(b) identifying one or more allele(s) at the one or more polymorphism(s) that are associated with increased WUE;
a method of producing a food or feed product under water-limited or drought conditions, the method comprising:
(a) obtaining a plant with reduced or abolished expression of at least one nucleic acid encoding a BLUS1 polypeptide and/or reduced or abolished activity of a BLUS1 polypeptide according to any method or plant as described herein;
(b) isolating a plant part or seed from the plant; and
(c) producing a food or feed product from the plant part or seed.
use of any plant as described herein for producing a food or feed product.
a method of quantifying stomatal blue light response and/or photosynthetic capacity in any one or more plants as described herein, wherein the method comprises:
(a) acclimatizing the one or more plants to red light conditions;
(b) obtaining one or more measurements under the red light conditions;
(c) subjecting the one or more plants to blue light conditions; and
(d) obtaining one or more further measurements under the blue light conditions.

### DETAILED DESCRIPTION OF EMBODIMENTS OF THE INVENTION

Embodiments of the present invention will now be described hereinafter, by way of example only, with reference to the accompanying drawings in which:

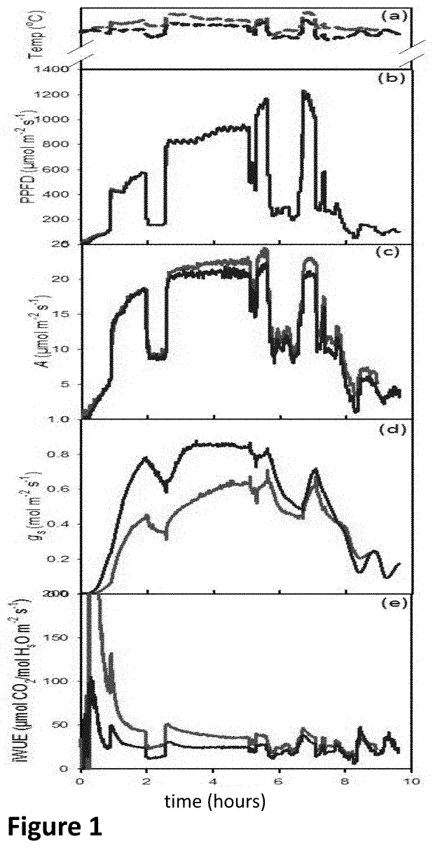

FIG. 1 shows diurnal gas exchange measurement of wheat taken with blue spectra included (blue lines) and without blue spectra (red lines). (a) leaf temperature; (b) light intensity; (c) photosynthesis; (d) stomatal conductance; (e) WUE over a 10 h diurnal.

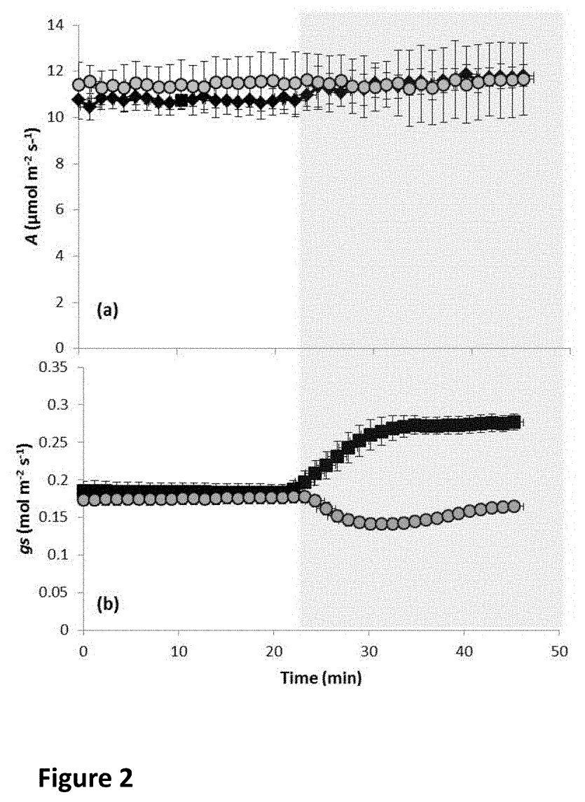

FIG. 2 shows effects of additional blue light (shaded area) follow acclimatisation to saturating red light (white area) on (a) assimilation rate and (b) stomatal conductance in wild type (WT) (black square) and Arabidopsis blus1 mutants (grey circles). The blus1 mutants show reduced gₛ in response to shift in red light to include blue. The reduction in gₛ does not affect A, so the mutant plants lose less water relative to carbon gain.

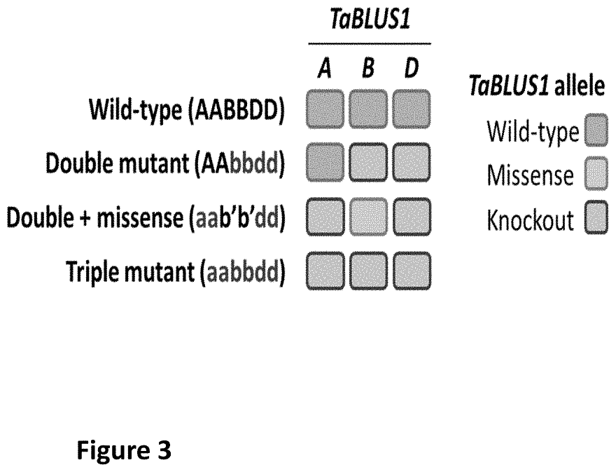

FIG. 3 shows the allelic combinations of TaBLUS1 generated in wheat (Cadenza).

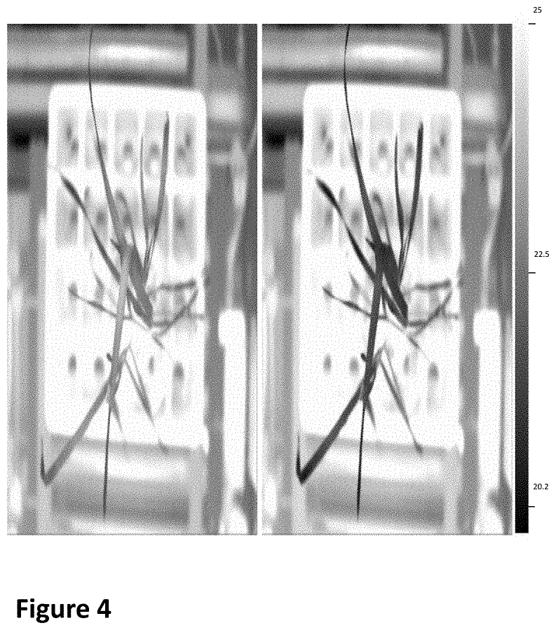

FIG. 4 shows a thermal screen to detect gₛ specific responses to blue light in wheat. The left image shows plant temperature after an exposition to 1000 μmol m⁻² s⁻¹ of red light for 1 h (to saturate photosynthesis and open stomata). The right image shows the same plants after 15 min additional low (15 μmol m⁻² s⁻¹) blue light. Stomata open and lower the leaf temperature but with no additional benefit on photosynthetic rate.

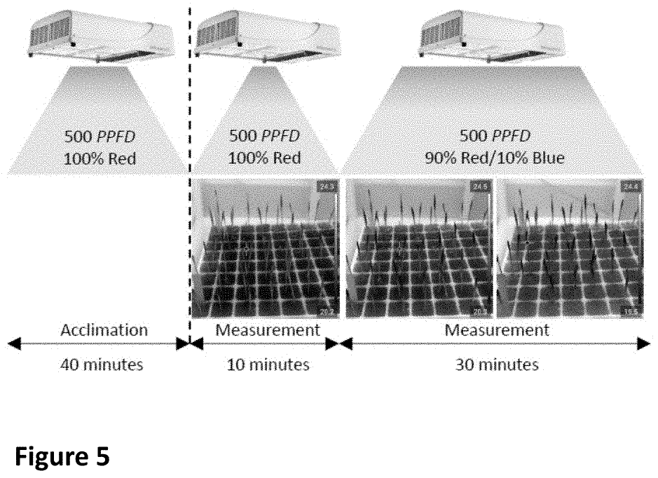

FIG. 5 shows high throughput screens to quantify stomatal BL responses and photosynthetic capacity in wheat mutants.

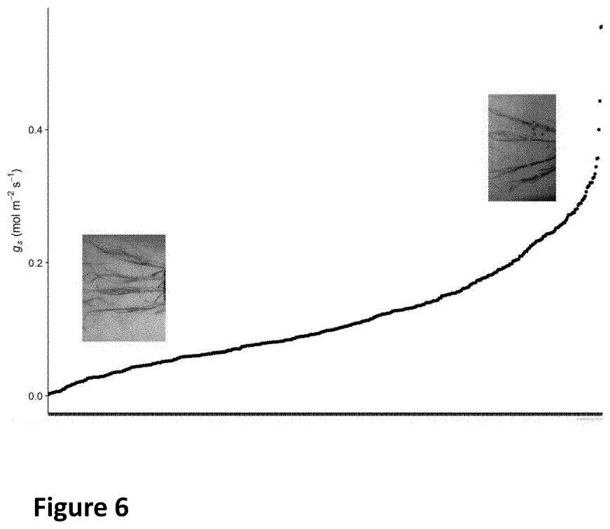

FIG. 6 shows the measurement of stomatal conductance used to identify single, double or triple mutant wheat lines.

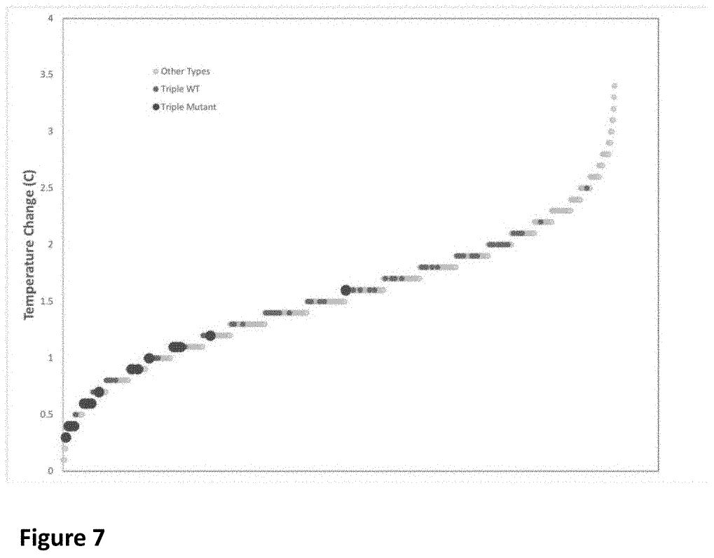

FIG. 7 shows the measurement of leaf temperature in various wheat lines. The triple deletion blus1 mutants in wheat (red dots) have a significantly smaller temperature range as compared to wild type.

### SEQUENCE LISTING

SEQ ID NO: 1 shows full length nucleic add sequence of BLUS1 in Arabidopsis thaliana
(At4g14480):

| | |
|---|---|
| GTTTGAAGTT GAAATGATAG TGTTTGCCCT TTGGGTCCCT GGAAAGACCA AAGGTAAAAT | 60 |
| CTCATTAACT AAGGTTAGTT AGTTAGCTCT TTCACATGCT TCATCTTATT GTTTCATCAA | 120 |
| CATATTTTTA AGTAAGAGAT AAGGGTTTTG CTTTTACATT AAAGGCTCTC TTTTTTTTCA | 180 |
| AGTTCACATG CTAAACCAAC TCTTTCTTTT TTCATCCCTA TATGAATAGC CTCATGGTAC | 240 |
| TATTAGCCTC TAAAAGACCA CAATTCCACT AAAAACATAA CAATTCAATA AGCAAGAGTG | 300 |
| TACTCATCTT CTTTCTATTT ATGGCTCGGA ACAAGCTCGA GTTCCCTCTT GATGCTGAAG | 360 |
| CCTACGAGAT CATCTGCAAG ATAGGCGTTG GTGTTAGTGC TTCGGTCTAC AAGGCCATAT | 420 |
| GCATTCCGAT GAACTCAATG GTAGTTGCTA TCAAAGCCAT CGATCTTGAT CAGTCGCGGG | 480 |
| CTGACTTTGA CAGTCTTCGC CGTGAAACCA AGACGATGTC TCTGCTTTCT CATCCGAATA | 540 |
| TTCTCAATGC TTATTGTTCA TTCACCGTTG ATCGATGTCT CTGGGTGGTT ATGCCATTCA | 600 |
| TGTCTTGTGG CTCTCTTCAT TCGATCGTCT CTTCGTCTTT TCCAAGTGGG TTACCAGAAA | 660 |
| ACTGCATTTC CGTCTTCCTC AAGGAAACTC TGAATGCAAT CTCGTATCTT CACGATCAGG | 720 |
| GTCATTTGCA CCGTGACATC AAGGCAGGTA ACATTCTGGT AGATTCTGAT GGATCCGTGA | 780 |
| AGCTCGCTGA TTTCGGAGTA TCTGCATCCA TCTATGAACC CGTGACATCT TCCTCTGGAA | 840 |
| CAACATCTTC TTCTTTAAGG TTAACTGATA TAGCGGGAAC ACCGTATTGG ATGGCTCCGG | 900 |
| AAGTGGTTCA TTCCCACACA GGGTATGGTT TCAAAGCAGA CATTTGGTCT TTCGGGATAA | 960 |
| CAGCGTTGGA GTTAGCACAT GGAAGACCTC CGTTATCTCA CTTACCGCCG TTGAAGAGTC | 1020 |
| TGCTCATGAA GATCACCAAA AGGTTTCATT TTTCTGATTA CGAGATCAAT ACGAGCGGAA | 1080 |
| GCAGCAAAAA GGGTAACAAG AAGTTCTCAA AAGCTTTTAG AGAAATGGTT GGTTTGTGTC | 1140 |
| TAGAGCAAGA TCCTACTAAA AGACCATCGG CAGAGAAGTT GTTGAAGCAT CCTTTTTTCA | 1200 |
| AGAACTGTAA AGGACTCGAC TTTGTGGTCA AGAACGTGTT GCATAGCTTG TCAAACGCAG | 1260 |
| AGCAGATGTT TATGGAGAGT CAGATTTTGA TCAAGAGTGT TGGAGATGAT GATGAAGAAG | 1320 |
| AAGAAGAAGA AGACGAAGAG ATAGTGAAGA ATAGAAGAAT CAGTGGGTGG AATTTCCGTG | 1380 |
| AAGACGATCT CCAACTTAGT CCAGTGTTCC CAGCTACTGA ATCAGACTCT TCTGAGTCCA | 1440 |
| GTCCACGTGA AGAAGATCAA TCAAAAGACA AAAAGGAAGA CGATAACGTC ACAATAACGG | 1500 |
| GGTATGAACT CGGTTTAGGT TTGTCGAACG AGGAAGCTAA GAACCAAGAA GGTGAGGTTG | 1560 |
| TTGGGTTTGA TAAAGATTTG GTGTTAGAGA AACTGAAAGT GTTGAAGAAA AGTTTAGAGC | 1620 |
| ATCAAAGAGC AAGAGTGTCG ATTATAATCG AAGCATTGAG TGGGGACAAG GAAGAGAAGA | 1680 |
| GCAGAGAAGA AGAGCTTCTA GAGATGGTGG AGAAGTTAAA GATTGAATTG GAAACTGAGA | 1740 |
| AGCTAAAGAC CTTGCGTGCT GATAAAGATA GTGTTTTGGG TTAACTATTC TAAACTTGTT | 1800 |
| AATATTTTTT TTCTATATGC TAAAATTATA TAAGTGGCAT GACATTACGA TCAATTGTTT | 1860 |
| TCAATTTAAC TCGTTTTATT TGTCAGTTTA GAATGGCTCC AAATTAAGTT TTGGTAAACA | 1920 |
| AGTTATATAT TCACCAAAAC AACATATAAA TATTTCCTC | 1959 |

SEQ ID NO: 2 shows the amino acid sequence of BLUS1 in Arabidopsis thaliana
(At4g14480):

| | |
|---|---|
| 1 | MARNKLEFPL DAEAYEIICK IGVGVSASVY= KAICIPMNSM VVAIKAIDLD |
| 51 | QSRADFDSLR RETKTMSLLS HPNILNAYCS FTVDRCLWVV MPFMSCGSLH |
| 101 | SIVSSSFPSG LPENCISVFL KETLNAISYL HDQGHLHRDI KAGNILVDSD |
| 151 | GSVKLADFGV SASIYEPVTS SSGTTSSSLR LTDIAGTPYW MAPEVVHSHT |
| 201 | GYGFKADIWS FGITALELAH GRPPLSHLPP LKSLLMKITK RFHFSDYEIN |
| 251 | TSGSSKKGNK KFSKAFREMV GLCLEQDPTK RPSAEKLLKH PFFKNCKGLD |
| 301 | FVVKNVLHSL SNAEQMFMES QILIKSVGDD DEEEEEEDEE IVKNRRISGW |
| 351 | NFREDDLQLS PVFPATESDS SESSPREEDQ SKDKKEDDNV TITGYELGLG |
| 401 | LSNEEAKNQE GEVVGFDKDL VLEKLKVLKK SLEHQRARVS IIIEALSGDK |
| 451 | EEKSREEELL EMVEKLKIEL ETEKLKTLRA DKDSVLG |

SEQ ID NO: 3 shows full length nucleic acid sequence of BLUS1 from genome A of wheat:

| | |
|---|---|
| ATGGCAGACG ACGCGGGGCC TGGCGGCGAG GCCAAGTACC CGCTCAACCC GGATTGCTAC | 60 |
| CGCCTGCTCT GCAAGATCGG GAGCGGCGTC AGCGCCGTCG TGTACAAGGC CGCGTGCCTG | 120 |
| CCGCTCGGCT CCGTGCCGGT GGCCATCAAG GCCATCGACC TCGAGCGCTC CCGCGCCAAC | 180 |
| CTGGAGGACG TGTGGCGGGA GGCCAAGGCC ATGGCGCTCC TGTCGCACGC CAACGTCCTG | 240 |
| CGCGCGCACT GCTCCTTCAC CGTGGGCAGC CACCTGTGGG TGGTCATGCC GTTCATGGCC | 300 |
| GCCGGCTCTC TGCACTCCAT CCTGGCCCAC GGCTTCCCCG ACGGCCTCCC GGAGCCGTGC | 360 |
| ATCGCGGTGG TGCTCAAGGA GACGCTCCGT GCGCTCTGCT ACCTTCACGA GCAGGGCCGC | 420 |
| ATCCACCGCG ACATCAAGGC CGGGAACGTC CTCGTCGACT CCGACGGCTC CGTCAAGCTC | 480 |
| GCCGACTTCG GCGTGTCCGC GTCCATATAC GAGACCCCGC CGCCGGCGTC GTCCTTCTCC | 540 |
| GGGCCCCTGA CCCACGCCCC CCAGGTTGTC CTCAGCTCTT CTTCCTACTT CAGCGAGATG | 600 |
| GCAGGGACGC CGTACTGGAT GGCGCCGGAG GTCATCCACT CGCACGTCGG CTACGGCATC | 660 |
| AAGGCCGACA TCTGGTCGTT CGGCATCACG GCCCTGGAGC TCGCGCACGG CCGGCCGCCC | 720 |
| CTCTCCCACC TGCCGCCGTC CAAGTCGATG CTGATGAGGA TCACGAGCCG CGTCCGGATG | 780 |
| GAGGACGCGG AGATCTCCAA GAACAAGAAG CTATCCAAGG CGTTCAAGGA CATGGTGTCC | 840 |
| TCCTGCCTGT GCCAAGAGCC GGCCAAGCGG CCGTCGGCGG AGAAGCTTCT CCGGCACCCG | 900 |
| TTCTTCAAAG GCTGCCGCTC CAAGGACTAC CTCGTCCGCA ACGTCCTCAG CGTCGTGCCG | 960 |
| AGCATCGAGG AGCGCTGCAA GGACGTCACG GGCCTCTGCG GCTGCGCCGC CGGGGGCGCG | 1020 |
| CGCTGCGTGT CGCCGTGCCA CGGCCAGGCG AGCGCGAGCA TCGTCAAGAA CCGCCGCATG | 1080 |
| AGCGGCTGGA ACTTCGGCGC GGACTGCCCG AGGAAGGAAG ACGCGGACAG CTTTGAGGAT | 1140 |
| CTCGACCAGA CTGAGACGGT AGCACGGCTG TTCCTTCCAC TCGACGACGA GGACACGGTG | 1200 |
| CCCGAGCGGG CCTGCGACGG GGCCGGAGAA GATGGAGACA AGGGAACAAT GGAGCAGCAA | 1260 |
| GGTGATCGAG AGGAAAACGA GGGATCGTTC GGCGTGAAAG GGGTGGTGGT GCCGCATCTG | 1320 |
| ATGACTATCT TGGGAAGCCT CGAGGTGCAG AAGAGGATGC TGGCCCAAGA ACTGGAAGGT | 1380 |
| GGCTGCTGCT ATCACCACGA CGGCAACTGC TGCCGCGAGA CGACGGCCAG GGAGGAGATG | 1440 |
| CTACTCGCGT ACGTGCGTCA GCTCGAGCAG AGGGTGGAGG TGCTGACCTT GGAGGTCGAG | 1500 |
| GAGGAAATCG CCAGGAATGC CCACCTGGAG GAGCTTCTTC GTGAGAGGGC TGGTTGA | 1557 |

SEQ ID NO: 4 shows the amino acid sequence of BLUS1 from genome A of wheat:

| | |
|---|---|
| 1 | MADDAGPGGE AKYPLNPDCY RLLCKIGSGV SAVVYKAACL PLGSVPVAIK |
| 51 | AIDLERSRAN LEDVWREAKA MALLSHANVL RAHCSFTVGS HLWVVMPFMA |
| 101 | AGSLHSILAH GFPDGLPEPC IAVVLKETLR ALCYLHEQGR IHRDIKAGNV |
| 151 | LVDSDGSVKL ADFGVSASIY ETPPPASSFS GPLTHAPQVV LSSSSYFSEM |
| 201 | AGTPYWMAPE VIHSHVGYGI KADIWSFGIT ALELAHGRPP LSHLPPSKSM |
| 251 | LMRITSRVRM EDAEISKNKK LSKAFKDMVS SCLCQEPAKR PSAEKLLRHP |
| 301 | FFKGCRSKDY LVRNVLSVVP SIEERCKDVT GLCGCAAGGA RCVSPCHGQA |
| 351 | SASIVKNRRM SGWNFGADCP RKEDADSFED LDQTETVARL FLPLDDEDTV |
| 401 | PERACDGAGE DGDKGTMEQQ GDREENEGSF GVKGVVVPHL MTILGSLEVQ |
| 451 | KRMLAQELEG GCCYHHDGNC CRETTAREEM LLAYVRQLEQ RVEVLTLEVE |
| 501 | EEIARNAHLE ELLRERAG |

SEQ ID NO: 5 shows full length nucleic acid sequence of BLUS1 from genome B of wheat:

| | |
|---|---|
| ATGGCAGACG AGGCGGGGGC TGGCGGCGAA GCCAAGTACC CGCTCAACCC AGAGTGCTAC | 60 |
| CGCCTGCTCT GCAAGATCGG GAGCGGCGTC AGCGCCGTCG TGTACAAGGC CGCGTGCCTG | 120 |
| CCGCTCGGCT CGGTGCCGGT GGCCATCAAG GCCATCGACC TCGAGCGTTC CCGCGCCAAC | 180 |
| CTGGAGGACG TGTGGCGGGA GGCCAAGGCC ATGGCGCTCC TGTCGCACGC CAACGTCCTG | 240 |
| CGCGCGCACT GCTCCTTCAC CGTGGGCAGC CACCTGTGGG TGGTCATGCC GTTCATGGCC | 300 |
| GCCGGCTCGC TGCACTCCAT CCTGGCCCAT GGCTTCCCCG ACGGCCTCCC GGAGCCGTGC | 360 |
| ATCGCGGTGG TACTCAAGGA GACGCTCCGT GCGCTCTGCT ACCTTCACGA GCAGGGCCGC | 420 |
| ATCCACCGCG ACATCAAGGC CGGGAACCTC CTCGTCGACT CTGACGGCTC CGTCAAGCTC | 480 |
| GCCGACTTCG GCGTGTCCGC GTCCATATAC GAGACCCCGC CGCCGGCGTC GTCCTTCTCC | 540 |
| GGGCCCTTGA CACACGCCCC TCAGGTTGTT CTCAGCTCTT CTTCCTACTT CAGCGAGATG | 600 |
| GCAGGGACGC CGTACTGGAT GGCGCCGGAG GTCATCCACT CGCACGTCGG CTACGGCATC | 660 |
| AAGGCCGACA TCTGGTCGTT CGGCATCACG GCCCTCGAGC TCGCGCACGG CCGGCCGCCC | 720 |
| CTCTCCCACC TGCCGCCGTC CAAGTCGATG CTGATGAGGA TCACGAGCCG CGTCCGGATG | 780 |
| GAGGACGCGG AGATCTCCAA GAACAAGAAG CTCTCCAAGG CGTTCAAGGC CATGGTGTCC | 840 |
| TCCTGCCTGT GCCAAGAGCC GGCCAAGCGG CCATCGGCGG AGAAGCTTCT CCGGCACCCG | 900 |
| TTCTTCAAAG GCTGCCGCTC CAAGGACTAC CTAGTGCGCA ACGTCCTCAG CATCGTGCCG | 960 |
| AGCATCGAGG AGCGCTGCAA GGACGTCACG GGCCTCTGCG GCTGCGCCGC CGGGGGCGCG | 1020 |
| CGCTGCGTGT CGCCGTGCCA CGGCCAGGCG AGCGCGAGCA TCGTCAAGAA CCGCCGAATG | 1080 |
| AGCGGCTGGA ACTTCGGCGC GGACTGCCCG AGAAAGGAAG ACGCGGACAG CTTTGAGGAG | 1140 |
| CTCGACCGGA CCGAGACGGT AGCACGGCTA TTCCTTCCAC TCGACGACGA GGACACCGTG | 1200 |
| CCCGAGCGGG CCTGCGAAGG CGCCGGAGAA GACGGAGATA AGGGAGTAAC GGAAGAACAA | 1260 |
| TGTGATCGAG AAGAAAACGA CGGATCGTTT GGCGTGAAAG GGGTGGTGGT GCCGCATCTG | 1320 |
| GTGACTATCT TGGGAAGCCT CGAGGTGCAG AAACGGATGC TGGCCCAAGA ACTGGAAGGT | 1380 |
| GGGTGCTGCT ATCACCACAA CGGCAACTGC TGCCGCGAGA CGACGGCCAG GGAGGAGATG | 1440 |
| CTACTCGCGT ACGTGCGTCA GCTCGAGCAG AGGGTGGAGG TGCTGACCCT GGAGGTAGAG | 1500 |
| GAGGAAATCA CCAGGAATGC CCACCTGGAG GAGCTTCTTC GTGGGAGGGC TGGTTGA | 1557 |

SEQ ID NO: 6 shows the amino acid sequence of BLUS1 from genome B of wheat:

| | |
|---|---|
| 1 | MADEAGAGGE AKYPLNPECY RLLCKIGSGV SAVVYKAACL PLGSVPVAIK |
| 51 | AIDLERSRAN LEDVWREAKA MALLSHANVL RAHCSFTVGS HLWVVMPFMA |
| 101 | AGSLHSILAH GFPDGLPEPC IAVVLKETLR ALCYLHEQGR IHRDIKAGNL |
| 151 | LVDSDGSVKL ADFGVSASIY ETPPPASSFS GPLTHAPQVV LSSSSYFSEM |
| 201 | AGTPYWMAPE VIHSHVGYGI KADIWSFGIT ALELAHGRPP LSHLPPSKSM |
| 251 | LMRITSRVRM EDAEISKNKK LSKAFKAMVS SCLCQEPAKR PSAEKLLRHP |
| 301 | FFKGCRSKDY LVRNVLSIVP SIEERCKDVT GLCGCAAGGA RCVSPCHGQA |
| 351 | SASIVKNRRM SGWNFGADCP RKEDADSFEE LDRTETVARL FLPLDDEDTV |
| 401 | PERACEGAGE DGDKGVTEEQ CDREENDGSF GVKGVVVPHL VTILGSLEVQ |
| 451 | KRMLAQELEG GCCYHHNGNC CRETTAREEM LLAYVRQLEQ RVEVLTLEVE |
| 501 | EEITRNAHLE ELLRGRAG |

SEQ ID NO: 7 shows full length nucleic acid sequence of BLUS1 from genome D of wheat:

| | |
|---|---|
| ATGGCAGACG ACGCGGGGGC CGGCGGCGAA GCCAAATACC CGCTCAACCC AGAGTGCTAC | 60 |
| CGCCTGCTCT GCAAGATCGG AAGCGGCGTC AGCGCCGTCG TGTACAAGGC CGCGTGCCTG | 120 |
| CCGCTCGGTT CCGTGCCGGT GGCCATCAAG GCCATCGACC TCGAGCGCTC CCGCGCCAAC | 180 |
| CTGGAGGACG TGTGGCGGGA GGCCAAGGCC ATGGCGCTCC TGTCGCACGC CAACGTCCTG | 240 |
| CGCGCGCACT GCTCCTTCAC GGTGGGCAGC CACCTATGGG TGGTCATGCC GTTCATGGCC | 300 |
| GCCGGCTCGC TGCACTCCAT CCTCGCCCAC GGCTTCCCCG ACGGCCTACC AGAGCCGTGC | 360 |
| ATCGCGGTGG TGCTCAAGGA GACGCTCCGT GCGCTCTGCT ACCTTCACGA GCAGGGCCGC | 420 |
| ATCCACCGCG ACATCAAGGC CGGAAACGTC CTCGTCGACT CCGACGGCTC CGTCAAGCTC | 480 |
| GCCGACTTCG GCGTGTCCGC GTCCATATAC GAGACCCCGC CGCCGGCGTC GTCCTTCTCC | 540 |
| GGGCCCTTGA CACACGCCCC TCAGATTGTC CTCAGCTCTT CTTCCTACTT CAGCGAGATG | 600 |
| GCAGGGACGC CGTACTGGAT GGCGCCGGAG GTCATCCACT CGCACGTCGG CTACGGCATC | 660 |
| AAGGCCGACA TCTGGTCGTT CGGCATCACG GCCCTGGAGC TCGCGCACGG CCGGCCGCCC | 720 |
| CTCTCCCACC TGCCGCCGTC CAAGTCGATG CTGATGAGGA TCACGAGCCG CGTCCGGATG | 780 |
| GAGGACGCGG AGATCTCCAA GAACAAGAAG CTCTCCAAGG CGTTCAAGGA CATGGTGTCC | 840 |
| TCCTGCCTGT GCCAAGAGCC GGCCAAGCGG CCATCGGCGG AGAAGCTTCT CCGGCATCCG | 900 |
| TTCTTCAAAG GCTGCCGCTC CAAGGACCAC CTCGTCCGCA ACGTCCTCAG CGTCGTGCCG | 960 |
| AGCATCGAGG AGCGCTGCAA GGACGTCACG GGCCTCTGCG GCTGCGCCGC CGGGGGCGCG | 1020 |
| CGCTGCGTGT CGCCGTGCCA CGGCCAGGCG AGCGCGAGCA TCGTCAAGAA CCGCCGCATG | 1080 |
| AGCGGCTGGA ACTTCGGCGC GGACTGCCCG AGGAAGGAAG ACGCGGACAG CTTTGAGGAG | 1140 |
| CTCGACCGGA CCGAGACGAC AGCACGGCTG TTCCTTCCAC TCGACGACGA GGACACCGTG | 1200 |
| CCTGAGCGGA CCTGCGAAGG CGCCGGAGAA GACGGAGATA AGGGAGTAAC GGAAGAACAA | 1260 |
| GGTGATCGAG AAGAAAACGA GGGATCGTTT GGCGTGAAAG GGGTGGTGGT GCCGCATCTG | 1320 |
| ATGACTATCT TGGGAAGCCT CGAGGTGCAG AAACGGATGC TGGCCCAAGA ACTGGAAGGT | 1380 |
| GGCTGCTGCT ATCACCACGA CGGCAACTGC TGCGGCGAGA CGACGGCCAG GGAGGAGATG | 1440 |
| CTACTCGCGT ACGTACGTCA GCTCGAGCAG AGGGTGGAGA TGCTGACCTT GGAGGTAGAG | 1500 |
| GAGGAAATCA CCAGGAATGC CCAGCTGGAG GAGCTTCTTC GTGAGAGGGC TGGTTAA | 1557 |

SEQ ID NO: 8 shows the amino acid sequence of BLUS1 from genome D of wheat:

| | |
|---|---|
| 1 | MADDAGAGGE AKYPLNPECY RLLCKIGSGV SAVVYKAACL PLGSVPVAIK |
| 51 | AIDLERSRAN LEDVWREAKA MALLSHANVL RAHCSFTVGS HLWVVMPFMA |
| 101 | AGSLHSILAH GFPDGLPEPC IAVVLKETLR ALCYLHEQGR IHRDIKAGNV |
| 151 | LVDSDGSVKL ADFGVSASIY ETPPPASSFS GPLTHAPQIV LSSSSYFSEM |
| 201 | AGTPYWMAPE VIHSHVGYGI KADIWSFGIT ALELAHGRPP LSHLPPSKSM |
| 251 | LMRITSRVRM EDAEISKNKK LSKAFKDMVS SCLCQEPAKR PSAEKLLRHP |
| 301 | FFKGCRSKDH LVRNVLSVVP SIEERCKDVT GLCGCAAGGA RCVSPCHGQA |
| 351 | SASIVKNRRM SGWNFGADCP RKEDADSFEE LDRTETTARL FLPLDDEDTV |
| 401 | PERTCEGAGE DGDKGVTEEQ GDREENEGSF GVKGVVVPHL MTILGSLEVQ |
| 451 | KRMLAQELEG GCCYHHDGNC CGETTAREEM LLAYVRQLEQ RVEMLTLEVE |
| 501 | EEITRNAQLE ELLRERAG |

Underlined sequences above represent example positions of specific amino acid mutations as described herein.

The practice of embodiments of the present invention employs, unless otherwise indicated, conventional techniques of chemistry, molecular biology, which are within the skill of those working in the art.

Most general chemistry techniques can be found in Comprehensive Heterocyclic Chemistry IF (Katritzky et al., 1996, published by Pergamon Press); Comprehensive Organic Functional Group Transformations (Katritzky et al., 1995, published by Pergamon Press); Comprehensive Organic Synthesis (Trost et al., 1991, published by Pergamon); Heterocyclic Chemistry (Joule et al. published by Chapman & Hall); Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure. For chemical terms, the skilled person may refer to the International Union of Pure and Applied Chemistry (IUPAC). Units, prefixes and symbols are denoted in their Systéme International d'Unités (SI) accepted form. Numeric ranges are inclusive of the numbers defining the range.

### Methods of Increasing Water Use Efficiency

In certain embodiments, the invention provides a method of increasing WUE in a plant. The term “WUE” is understood as the ratio of CO₂ uptake to water loss. WUE can be calculated as the ratio of biomass produced per unit of water transpired during plant growth. Instantaneous measurements of WUE can be obtained as the ratio of carbon dioxide assimilation to transpiration using gas exchange measurements. In certain embodiments, WUE may be obtained as the ratio between the amounts of biomass produced per unit water transpired as measured gravimetrically and the ratio of photosynthetic rate to the rate of transpiration as measured using gas exchange quantification of a leaf or shoot of the plant.

In certain embodiments, the plant has increased WUE relative to a control or wild-type plant. For example, the plant may have increased WUE as compared to plants with normal or unaltered levels of BLUS1 and grown under similar or the same conditions. The plant may have increased WUE as compared to a reference value obtained from a control or wild-type plant.

In certain embodiments, the plant has an at least 10%, 15%, 20%, 25%, 30%, 35%, 40%, 45%, 50%, 55%, 60%, 70% or more increase in WUE as compared to a control or wild-type plant.

In certain embodiments, the plant has an increased leaf temperature as compared to a control or wild-type plant. For example, the plant may comprise leaves with an increased temperature of at least about 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.0° C., 2.0° C., 2.1° C., 2.2° C., 2.3° C., 2.4° C., 2.5° C. or more. Typically, the plant comprises leaves with an increased temperature of about 1° C.-2° C. as compared to a control or wild-type plant.

A control or wild-type plant may be a plant which has not been modified according to the methods of the invention. For example, the control or wild type plant may have normal (e.g. unaltered) expression of BLUS1 and/or activity of BLUS1. Typically, the control or wild-type plant is of the same plant species, preferably having the same genetic background as the modified plant.

In certain embodiments, increased WUE efficiency results from an about 5%, 10%, 15%, 20%, 25%, 30%, 35%, 40%, 45%, 50%, 55%, 60% or more reduction in stomatal conductance (gₛ) under natural light. Typically, the reduction in gₛ is relative to a control or wild-type plant as described herein.

As used herein, the term “natural light” refers to the full spectrum of photosynthetically active radiation (PAR) e.g. light wavelengths from about 400 nm to about 700 nm.

As used herein, the term “blue light” refers to the blue wavelengths of PAR e.g. light wavelengths from about 400 nm to about 500 nm.

As used herein, the term “red light” refers to the red wavelengths of PAR e.g. light wavelengths from about 600 nm to about 700 nm.

Stomatal conductance (usually measured in in mmol m⁻² s⁻¹), is the measure of the rate of passage of carbon dioxide entering, or water vapor exiting through the stomata of a leaf. Any suitable method may be used to measure gₛ including the methods described further herein.

As used herein, the term “plant” refers to all genera and species of higher and lower plants of the Plant Kingdom. The term includes the mature plants, seeds, shoots, seedlings, and part, propagation material, plant organ tissue, protoplasts, callus and other cultures, for example cell cultures, derived from them, and all other species of groups of plant cells giving functional or structural units. Mature plants refers to plants at any developmental stage beyond the seedling. Seedling refers to a young, immature plant at an early developmental stage.

In certain embodiments, the plant is a monocot or a dicot.

In certain embodiments, the plant is a model species such as A. thaliana.

In certain embodiments, the plant is a crop plant. A crop plant is any plant which is grown on a commercial scale for human or animal consumption or use.

In certain embodiments, the crop plant is a C₃ plant.

In certain embodiments, the plant (e.g. crop) is temperature tolerant (e.g. heat tolerant). In other words, the plant may be tolerant to an increase in leaf temperature of about 0.5° C., 1° C., about 2° C. or more. Temperature (e.g. heat) tolerant crops have been developed in a range of crop species and are commercially available.

In certain embodiments, the plant is a cereal crop such as Triticum species (e.g. wheat), Oryza sativa (e.g. rice), Glycine max (e.g. soybean), barley, rye, oats, sorghum, alfalfa, clover, Zea mays (maize) and the like. The plant may be an oil-producing plant such as canola, safflower, sunflower, peanut, cacao and the like. The plant may be tobacco. The plant may be a vegetable crop such as tomato, potato, pepper, eggplant, sugar beet, carrot, cucumber, lettuce or pea. The plant may be a Brassica species such as B. campestris, B. napus, B. rapa or B. carinata. The plant may be Cannabis sativa (e.g. hemp), Carthamus tinctorius (e.g. safflower), Linum usitatissimum (e.g. linseed or flax) or Olea europaea (olive).

In certain embodiments, the plant is wheat. For example, the wheat may be a hexaploid species such as T. aestivum or T. spelta. The wheat may be a tetraploid species such as T. Durum or T. dicoccon. Typically, the wheat is hexaploid.

In certain embodiments, the wheat comprises a Cadenza, Reedling, Kingbird or Sokoll background.

In certain embodiments, the method of increasing WUE in the plant comprises reducing or abolishing the expression of at least one nucleic acid sequence encoding a BLUS1 polypeptide.

The term “reducing” means a decrease in the levels of BLUS1 expression and/or BLUS1 activity (e.g. biological activity) by up to about 10%, 20%, 30%, 40%, 50%, 60%, 70%, 80%, 90% or more. The term “abolishing” means that substantially no or no expression of BLUS1 is detectable and/or that substantially no or no functional BLUS1 polypeptide is produced. Any method for determining the level and/or activity of BLUS1 may be employed, as well known to the skilled person.

The nucleic acid sequence may be any sequence associated with expression of BLUS1.

In certain embodiments, the nucleic acid sequence is a coding sequence. For example, the nucleic acid sequence may be genomic DNA (e.g. including exons and/or introns).

In certain embodiments, the nucleic acid sequence is mRNA or cDNA.

In certain embodiments, the nucleic acid sequence is non-coding regulatory sequence. For example, the non-coding regulatory sequence may be a promoter of BLUS1. A promoter of BLUS1 may comprise nucleic acid sequence extending for at least about 2 kbp, 3 kbp, 4 kbp, 5 kbp or more upstream of the ATG codon of the BLUS1 open reading frame. The non-coding regulatory sequence may be an enhancer of BLUS1 expression.

In certain embodiments, the nucleic acid sequence comprises SEQ ID NO: 1, 3, 5, 7 or a homolog or variant thereof as described herein.

In certain embodiments, the nucleic acid sequence encodes the amino acid sequences set forth in SEQ ID NO: 2, 4, 6, 8 or a homolog or variant thereof as described herein.

In certain embodiments, the nucleic acid sequence is a homolog of A. thaliana BLUS1 as set forth in SEQ ID NO: 1.

In certain embodiments, the nucleic acid sequence is a homolog of wheat BLUS1 as set forth in SEQ ID NO: 3, 5 or 7. For example, the nucleic acid sequence may be a homolog of wheat BLUS1 from genome A as set forth in SEQ ID NO: 3. The nucleic acid sequence may be a homolog of wheat BLUS1 from genome B as set forth in SEQ ID NO: 5. The nucleic acid sequence may be a homolog of wheat BLUS1 from genome D as set forth in SEQ ID NO: 7.

As used herein, the term “homolog” refers to a polypeptide or polynucleotide sequence possessing a high degree of sequence relatedness to a subject sequence. Such relatedness may be quantified by determining the degree of identity and/or similarity between the sequences being compared.

In certain embodiments, the nucleic acid sequence is at least about 50%, 60%, 70%, 80%, 90%, 95%, 96%, 97%, 98%, 99% or 100% identical to SEQ ID NO: 1, 3, 5 or 7.

In certain embodiments, the nucleic acid sequence encodes an amino acid at least about 50%, 60%, 70%, 80%, 90%, 95%, 96%, 97%, 98%, 99% or 100% identical to SEQ ID NO: 2, 4, 6 or 8.

In certain embodiments, the invention provides a method of increasing WUE by reducing or abolishing the activity of a BLUS1 polypeptide in the plant.

In certain embodiments, the BLUS1 polypeptide comprises the amino acid sequences set forth in SEQ ID NO: 2, 4, 6 or 8 or a homolog or variant thereof as described herein.

In certain embodiments, the amino acid has at least about 50%, 60%, 70%, 80%, 90%, 95%, 96%, 97%, 98%, 99% or 100% identity to SEQ ID NO: 2, 4, 6 or 8.

In certain embodiments, the amino acid sequence is a homolog of A. thaliana BLUS1 as set forth in SEQ ID NO: 2.

In certain embodiments, the amino acid sequence is a homolog of wheat BLUS1 as set forth in SEQ ID NO: 4, 6 or 8. For example, the nucleic acid sequence may be a homolog of wheat BLUS1 from genome A as set forth in SEQ ID NO: 4. The nucleic acid sequence may be a homolog of wheat BLUS1 from genome B as set forth in SEQ ID NO: 6. The nucleic acid sequence may be a homolog of wheat BLUS1 from genome D as set forth in SEQ ID NO: 8.

As used herein, the term “percent sequence identity” is the percentage of nucleic acids or amino acids that are identical when the two sequences are compared. Homology or sequence identity of two nucleic or amino acid sequences may be determined by methods known in the art. For example, the sequence identity may be determined using the algorithm of Karlin and Altschul (1990) Proc. Natl. Acad. Sci., U.S.A 87: 2264-2268. Such an algorithm is incorporated into the NBLAST and XBLAST programs of Altschul et al. (1990) J. Mol. 215: 403-410. BLAST nucleotide searches are performed with the NBLAST program, score=100, word length 12, to obtain nucleotide sequences homologous to a nucleic acid molecules of certain embodiments of the present invention. BLAST protein sequences are performed with the XBLAST program, score=50, word length=3, to obtain amino acid sequences homologous to a reference polypeptide (e.g. SEQ. ID. No 1). To obtain gapped alignments for comparison purposes, Gapped BLAST is utilised as described in Altshul et al. (1997) Nucleic Acids Res. 25: 3389-3402. When utilising BLAST and Gapped BLAST programs, the default parameters are typically used. (See http://www.ncbi.nlm.nih.gov).

Suitable homologues can be identified by sequence comparison and identification of conserved domains. For example, a suitable BLUS1 homolog may comprise a conserved serine/threonine protein kinase domain. There are predictors in the art that can be used to identify such sequences. The function of the homologue can be identified using methods described herein and a skilled person would thus be able to confirm the function. For example, the homolog may be overexpressed in a plant to confirm function.

In certain embodiments, the nucleotide sequences described herein (e.g. SEQ ID NO: 1, 3, 5 or 7) may be used to isolate corresponding sequences from other plants, for example other crops. For example, PCR, hybridization or other techniques may be used to identify corresponding sequences based on their sequence homology to the sequences described herein.

In certain embodiments, topology of the sequences and/or characteristic domains (e.g. serine/threonine protein kinase domain) may be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

In certain embodiments, homologous sequences may be confirmed by hybridization, wherein the hybridization takes place under stringent conditions. Using the stringent hybridization (i.e. washing the nucleic acid fragments twice where each wash is at room temperature for 30 minutes with 2× sodium chloride and sodium citrate (SCC buffer; 1.150. mM sodium chloride and 15 mM sodium citrate, pH 7.0) and 0.1% sodium dodecyl sulfate (SDS); followed by washing one time at 50° C. for 30 minutes with 2× SCC and 0.1% SDS; and then washing two times where each wash is at room temperature for 10 minutes with 2× SCC), homologous sequences can be identified comprising at most about 25 to about 30% base pair mismatches, or about 15 to about 25% base pair mismatches, or about 5 to about 15% base pair mismatches.

### Introducing Mutations into BLUS1

The present invention relates in part to the inventor's insight that loss of BLUS1 reduces water loss without compromising photosynthesis. For example, data described herein using Arabidopsis blus1 mutants shows that removing BLUS1 reduces stomatal responses to blue light leading to reduced aperture. Moreover, removing the blue light from the illumination spectra in wheat gives rise to similar effects. Based on this insight, the skilled person would be able to put the invention into effect in crop species following the methods described herein by targeting BLUS1 expression and/or activity.

In certain embodiments, the methods described herein comprise introducing at least one mutation into at least one nucleic acid sequence encoding BLUS1.

In certain embodiments, the methods described herein comprise introducing at least one mutation into at least one nucleic acid sequence encoding BLUS1 in wheat. For example, the method may comprises introducing at least one mutation into at least one nucleic acid sequence encoding BLUS1 in genome A, B and/or D of wheat.

As discussed herein, the nucleic acid sequence may comprise genomic DNA, mRNA, cDNA or non-coding regulatory sequence such as a promoter of BLUS1.

In certain embodiments, mutation(s) are introduced into a nucleic acid sequence of SEQ ID NO: 1, 3, 5, 7 or a homolog and/or variant thereof.

In certain embodiments, mutation(s) are introduced into a nucleic acid sequence encoding an amino acid sequence of SEQ ID NO: 2, 4, 6, 8 or a homolog and/or variant thereof.

In certain embodiments, the mutation(s) are loss of function mutations such as insertions, deletions or substitutions (e.g. single nucleotide polymorphisms).

In certain embodiments, a mutation may be introduced in the C-terminus of BLUS1.

In certain embodiments, a mutation may be introduced in the N-terminus of BLUS1.

In certain embodiments, one or more mutations may be “missense” mutations. A missense mutation is a change in the nucleic acid sequence that results in the substitution of an amino acid for another amino acid.

In certain embodiments, one or more mutations are “nonsense” mutations. A nonsense (or “STOP codon”) mutation is a change in the nucleic acid sequence that results in the introduction of a premature STOP codon and, thus, the termination of translation (resulting in a truncated protein). For example, any nucleotide substitution, insertion or deletion resulting in a “TGA” (“UGA” in RNA), “TAA” (“UAA” in RNA) or “TAG” (“UAG” in RNA) codon in the reading frame of the mature mRNA being translated will terminate translation.

In certain embodiments, one or more mutations are “insertion” mutations. An insertion mutation results from one or more nucleotides being added to the nucleic acid sequence.

In certain embodiments, one or more mutations are “deletion” mutations. A deletion mutation results from one or more nucleotides being deleted from the nucleic acid sequence.

In certain embodiments, one or more mutations are “frameshift” mutations. A frameshift mutation results from the nucleic acid sequence being translated in a different frame downstream of the mutation. A frameshift mutation can have various causes, such as the insertion or deletion of one or more nucleotides.

In certain embodiments, one or more mutations are “splice-site” mutations. A splice-site mutation results from the insertion, deletion or substitution of a nucleotide at the site of splicing.

In certain embodiments, one or more mutations are in a non-coding regulatory sequence (e.g. in a promoter of BLUS1). A mutation in a regulatory sequence may be a change of one or more nucleotides compared to the wild type sequence, e.g. by replacement, deletion or insertion of one or more nucleotides. For example, such a mutation may lead to reduced or no mRNA transcript of the gene being made.

In certain embodiments, the mutation may result in a substitution in a nucleic acid sequence corresponding to SEQ ID NO:1 or any homolog thereof. In certain embodiments, the mutation may result in a substitution in an amino acid sequence corresponding to SEQ ID NO: 2 or any homolog thereof. For example, the mutation may result in one or more of the following:
a substitution of D to N in an amino acid sequence corresponding to position 157 of SEQ ID NO: 2;
a substitution of A to T in an amino acid sequence corresponding to position 192 of SEQ ID NO: 2;
a substitution of E to K in an amino acid sequence corresponding to position 194 of SEQ ID NO: 2; and/or
a substitution of S to A in an amino acid sequence corresponding to position 348 of SEQ ID NO:2.

In certain embodiments, the mutation may result in a substitution in a nucleic acid sequence corresponding to SEQ ID NO: 3 or any homolog thereof. In certain embodiments, the mutation may result in a substitution in an amino acid sequence corresponding to SEQ ID NO: 4 or any homolog thereof. For example, the mutation may result in one or more of the following:
a substitution of C to T in a nucleic acid sequence corresponding to position 412 of SEQ ID NO: 3;
a substitution of Q to STP (e.g. Stop codon) in an amino acid sequence corresponding to position 138 of SEQ ID NO: 4;
a substitution of C to T in a nucleic acid sequence corresponding to position 1147 of SEQ ID NO: 3; and/or
a substitution of Q to STP (e.g. Stop codon) in an amino acid sequence corresponding to position 383 of SEQ ID NO: 4.

In certain embodiments, the mutation may result in a substitution in a nucleic acid sequence corresponding to SEQ ID NO: 5 or any homolog thereof. In certain embodiments, the mutation may result in a substitution in an amino acid sequence corresponding to SEQ ID NO: 6 or any homolog thereof. For example, the mutation may result in one or more of the following:
a substitution of C to T in a nucleic acid sequence corresponding to position 1258 of SEQ ID NO: 5; and/or
a substitution of Q to STP (e.g. Stop codon) in an amino acid sequence corresponding to position 420 of SEQ ID NO: 6.

In certain embodiments, the mutation may result in a substitution in a nucleic acid sequence corresponding to SEQ ID NO: 7 or any homolog thereof. In certain embodiments, the mutation may result in a substitution in an amino acid sequence corresponding to SEQ ID NO: 8 or any homolog thereof. For example, the mutation may result in one or more of the following:
a substitution of C to T in a nucleic acid sequence corresponding to position 497 of SEQ ID NO: 7;
a substitution of S to F in an amino acid sequence corresponding to position 166 of SEQ ID NO: 8;
a substitution of G to A in a nucleic acid sequence corresponding to position 770 of SEQ ID NO: 7; and/or
a substitution of R to H in an amino acid sequence corresponding to position 257 of SEQ ID NO: 8.

In certain embodiments, the mutation may result in a deletion of one or more nucleic acids (e.g. deletion of about 10, 20, 30, 50, 100 or more nucleic acids) from SEQ ID NO: 1, 3, 5 or 7. In certain embodiments, the mutation may result in a deletion of amino acids (e.g. deletion of about 10, 20, 30, 50, 100 or more amino acids) from SEQ ID NO: 2, 3, 6 or 8.

In certain embodiments, the mutation is introduced using mutagenesis (e.g. directed mutagenesis). Methods for mutagenesis in plants are well known in the art. Such technical processes lead to plants of the invention having modified genetic characteristics.

In certain embodiments, the mutagenesis is physical mutagenesis. For example, ultraviolet radiation, X-rays, gamma rays, fast or thermal neutrons or protons may be applied to the plant or seed thereof. The targeted population can then be screened to identify a blus1 loss of function mutant.

In certain embodiments, the mutagenesis is chemical mutagenesis. For example, the chemical may be ethyl methanesulfonate (EMS), methylmethane sulfonate (MMS), N-ethyl-N-nitrosurea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro- Nitrosoguanidine (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde.

In certain embodiments, the mutation is introduced using insertional mutagenesis. For example, transposons, site-directed nucleases (SDNs) or T-DNA mutagenesis may be used to reduce or abolish BLUS1 expression and/or BLUS1 activity.

In certain embodiments, the mutation is introduced by genome editing. Methods for genome editing in plants are well known in the art.

Any suitable method of genome editing may be used. For example, zinc finger nucleases (ZFNs), transcription activator-like effector nucleases (TALENs) or homing meganucleases may be used to edit the nucleic acid sequence encoding the BLUS1 polypeptide.

In certain embodiments, one or more nucleases from the bacterial adaptive immune system (e.g. Type II) are used to edit the nucleic acid sequence encoding the BLUS1 polypeptide. For example, CRISPR-associated proteins (e.g. Cas9, Cpf1, C2c1, C2c2, C2c3, Cas3, Cas3-HD, Cas 5, Cas7, Cas8, Cas10, or combinations or complexes thereof) may be used. In such embodiments, guide sequences may be used to target the nucleic acid sequence encoding the BLUS1 polypeptide. The use of CRISPR gene-editing technology in genome editing is well described in the art.

In certain embodiments, a crRNA (CRISPR RNA) and tracrRNA (trans-encoding CRISPR RNA) is used to guide the Cas endonuclease (e.g. Cas9) to at least one nucleic acid encoding BLUS1. Typically, the crRNA contains a spacer region complementary to one strand of the double strand DNA target and a region that base pairs with the tracrRNA (trans-encoding CRISPR RNA) forming a RNA duplex that directs the Cas endonuclease to cleave the DNA target.

In certain embodiments, a single guide RNA (sgRNA) is used to guide the Cas endonuclease (e.g. Cas9) to at least one nucleic acid encoding BLUS1. sgRNA is a synthetic RNA chimera created by fusing crRNA with tracrRNA. The sgRNA guide sequence located at its 5′ end may confer DNA target specificity. In plants, sgRNAs have been expressed using plant RNA polymerase III promoters, such as U6 and U3. Thus, sgRNA may be designed to target the nucleic acid sequence encoding the BLUS1 polypeptide.

In certain embodiments, the mutation is identified by a targeting induced local lesions in genomes (TILLING) method. TILLING has been applied in many plant species, including tomato (http://tilling.ucdavis.edu/index.php/Tomato Tilling), rice (Till et al. 2007, BMC Plant Biol 7: 19), Arabidopsis (Till et al. 2006, Methods Mol Biol 323: 127-35), Brassica, maize (Till et al. 2004, BMC Plant Biol 4: 12) and wheat (Rakszegi et al. 2010, Euphytica 174, 409-421).

TILLING populations in crops such as wheat are commercially available. For example, an EMS-mutagenized population of bread wheat cv. Candenza was developed and characterized by Dr. Andy Phillips at Rothamsted Research and Dr. Cristobal Uauy at John Innes Centre (JIC).

In certain embodiments, a mutation in a natural population is identified by EcoTILLING (see Till et al. 2006 (Nat Protoc 1: 2465-77) and Comai et al. 2004 (Plant J 37: 778-86). In certain embodiments, a TILLING method as described herein excludes EcoTILLING.

In TILLING, seeds may be mutagenized with chemical mutagens such as EMS. The resulting M1 plants may be self-fertilized and the M2 generation of individuals used to prepare DNA samples for mutational screening. DNA samples may be pooled and arrayed on microtiter plates and subjected to gene specific PCR.

The PCR amplification products may be screened for mutations in the BLUS1 target gene using any method that identifies heteroduplexes between wild type and mutant genes. Suitable methods include, for example, denaturing high pressure liquid chromatography (dHPLC), constant denaturant capillary electrophoresis (CDCE), temperature gradient capillary electrophoresis (TGCE) or by fragmentation using chemical cleavage. Preferably the PCR amplification products are incubated with an endonuclease that preferentially cleaves mismatches in heteroduplexes between wild type and mutant sequences. Cleavage products may be electrophoresed using an automated sequencing gel apparatus, and gel images analyzed with the aid of a standard commercial image-processing program.

Any primer specific to the BLUS1 nucleic acid sequence may be utilized to amplify the BLUS1 nucleic acid sequence within the pooled DNA sample. Preferably, the primer is designed to amplify the regions of the BLUS1 gene where useful mutations are most likely to arise, specifically in the areas of the BLUS1 gene that are highly conserved and/or confer activity (e.g. the kinase domain). To facilitate detection of PCR products on a gel, the PCR primer may be labelled using any conventional labelling method.

In certain embodiments, rapid high-throughput screening procedures may be used to identifying one or more mutations in BLUS1. Once a mutation is identified, the seeds of the M2 plant carrying that mutation may be grown into adult M3 plants and screened for the phenotypic characteristics associated with loss of function of blus1.

In the embodiments described above, one or more mutations are introduced by genetic engineering techniques. The plants (or parts or seed thereof) are not exclusively obtained by essentially biological processes, e.g. solely based on generating plants by traditional breeding methods.

The BLUS1 polypeptide is specifically localized in guard cells. Advantageously, genetic engineering techniques such as those described above (e.g. TILLING) may therefore achieve tissue specific response without requiring the use of transgenes.

In certain embodiments, the expression of BLUS1 and/or activity of BLUS1 may be reduced or abolished at the level of transcription or translation. For example, expression of BLUS1 nucleic acids can be reduced or abolished using gene silencing methods. “Silencing” refers to a down-regulation or complete inhibition of gene expression of the target gene or gene family. Methods for gene silencing in plants are well known in the art.

In certain embodiments, RNA interference, miRNA suppression, sense suppression, antisense suppression, virus-induced gene silencing or ribozymes are used to reduce or abolish the expression of at least one nucleic acid encoding a BLUS1 polypeptide.

In certain embodiments, short interfering RNA (siRNA), double-stranded RNA (dsRNA), micro-RNA (miRNA), antagomirs or short hairpin RNA (shRNA) capable of mediating RNA interference are used to reduce or abolish the expression of at least one nucleic acid encoding a BLUS1 polypeptide. The inhibition of expression of BLUS1 and/or BLUS1 activity can be measured by determining the presence and/or amount of the Blus1 transcript using techniques well known to the skilled person (e.g., Northern Blotting, RT-PCR or the like).

In one embodiment, RNA interference (RNAi) is used to reduce transcript levels of BLUS1 in the plant. The technique of RNAi in plants is well described in the art.

In one embodiment, antisense RNA is used to reduce transcript levels of BLUS1 in the plant. In this method, RNA silencing does not affect the transcription of BLUS1, but only causes sequence-specific degradation of target mRNAs. An “antisense” nucleic acid sequence comprises a nucleotide sequence that is complementary to at least a portion of the “sense” nucleic acid sequence encoding a BLUS1 polypeptide. The use of antisense RNAi in plant gene silencing is well described in the art.

In one embodiment, artificial and/or natural microRNAs (miRNAs) may be used to reduce or abolish BLUS1 transcription and/or mRNA translation. MicroRNAs (miRNAs) miRNAs are typically single stranded small RNAs typically 19-24 nucleotides long. Artificial microRNA (amiRNA) technology has been applied in plants to efficiently silence target genes of interest. The design principles for amiRNAs have been generalized and integrated into a Web-based tool (http://wmd.weigelworld.org). The use of miRNAs in plant gene silencing is well described in the art.

In one embodiment, a transgene is used to reduce or abolish the expression of BLUS1 and/or activity of BLUS1. Typically, the transgene has a similar sequence to BLUS1. This sequence homology may involve promoter regions or coding regions of BLUS1. When coding regions are involved, the transgene may have been constructed with a promoter that would transcribe either the sense or the antisense orientation of the coding sequence RNA.

In certain embodiments, the RNA or co-suppression molecule comprises a fragment of least about 17, 18, 19, 20, 25, 30 or 35 nucleotides (e.g. about 22 to 26 nucleotides) based on any nucleic acid sequence described herein (e.g. SEQ ID NOs 1, 3, 5, 7 or variants thereof).

Guidelines for designing effective siRNAs are known to the skilled person. In preferred embodiments, the criteria for choosing a sequence fragment from the target gene mRNA to be a candidate siRNA molecule may include one or more of: (i) a sequence from the target gene mRNA that is at least about 50-100 nucleotides from the 5′ or 3′ end of the native mRNA molecule, (ii) a sequence from the target gene mRNA that has a G/C content of between about 30% and 70%, (iii) a sequence from the target gene mRNA that does not contain repetitive sequences, (iv) a sequence from the target gene mRNA that is accessible in the mRNA, (v) a sequence from the target gene mRNA that is unique to BLUS1, (v) a sequence that avoids regions within 75 bases of a start codon. Software prediction programs are available that design optimal oligonucleotides (e.g. dsRNA) based on the above criteria. The optimized oligonucleotides may then be chemically synthesized and provided by commercial suppliers.

In certain embodiments, the RNA or co-suppression molecule (e.g. transgene) is introduced into the plant using conventional methods. Transformation methods in plants are well described in the art. For example, the RNA or co-suppression molecule may be introduced into the plant using a vector and/or Agrobacterium-mediated transformation. Stably transformed plants may be generated and the expression of the BLUS1 gene compared to wild type or control plants.

In certain embodiments, the method comprises reducing abolishing BLUS1 activity in the plant. For example, antibodies or aptamers may be raised against the polypeptide. Such antibodies or aptamers, may, for example, reduce or abolish BLUS1 activity through disrupting protein signalling or in other ways. Thus, antibodies or aptamers may be used to reduce or abolish BLUS1 polypeptide activity in guard cells of plants. The raising of antibodies or aptamers against target polypeptides in plants is well described in the art.

### Plants with Increased WUE

In certain embodiments, the invention provides plants having reduced or abolished expression of at least one nucleic acid encoding a BLUS1 polypeptide and/or reduced or abolished activity of a BLUS1 polypeptide.

The plant may be any plant (or part thereof) as described herein. For example, the plant may be a crop plant. Typically, the crop plant is a C3 plant such as wheat.

In certain embodiments, the plant is genetically engineered or modified as described herein. Typically, the plant is not exclusively obtained by an essentially biological process.

In certain embodiments, the plant is genetically altered compared to a naturally occurring wild type plant. For example, the plant may have been altered compared to a wild type plant using any mutagenesis method described herein.

In certain embodiments, the plant is transgenic. For example, the plant may comprise a transgenic construct. The transgenic construct may reduce or abolish the expression of at least one nucleic acid encoding a BLUS1 polypeptide. For example, the transgenic construct may lead to RNA interference, sense suppression, antisense suppression and/or miRNA suppression as described herein.

In certain embodiments, the plant has increased WUE as described herein.

In certain embodiments, the plant has increased leaf temperature as described herein.

In certain embodiments, the plant has reduced stomatal conductance (gₛ) as described herein.

In certain embodiments, the plant has increased drought resistance, biomass and/or yield as described herein.

In certain embodiments, one or more mutations are introduced into the nucleic acid sequence encoding a BLUS1 polypeptide as described herein. For example, mutation(s) may be introduced into coding or non-coding regulatory sequence of BLUS1.

In certain embodiments, one or more mutations are introduced into a least one plant cell and the plant is regenerated from the at least one mutated plant cell.

In certain embodiments, the plant comprises one or more blus1 alleles.

In certain embodiments, the invention provides a plant (or part thereof) obtainable by any method described herein.

In certain embodiments, the plant comprises one or more transgenic constructs. For example, the plant may comprise a construct (or vector) expressing RNA or a co-suppression molecule (e.g. transgene) that targets BLUS1 expression as described herein. Typically, the construct is stably incorporated into the plant genome.

In certain embodiments, the plant is or has been selected for further propagation. The selected plants may be propagated by any suitable technique such as clonal propagation or classical breeding techniques. For example, a first generation (or T1) transformed plant may be selfed and homozygous second-generation (or T2) transformants selected. The T2 plants may then further be propagated through classical breeding techniques.

In certain embodiments, the invention provides a harvestable part of the plant (e.g. seed). Typically, the harvestable part of the plant (e.g. seed) comprises a mutation or transgenic construct as described herein.

### Methods of Identifying Alleles Associated with Increased WUE

In certain embodiments, the invention provides a method of identifying one or more alleles associated with increased WUE in one or more plants.

In certain embodiments, the invention provides a method of screening a population of plants.

In certain embodiments, the method comprises detecting in the plant(s) one or more polymorphism(s) in a nucleic acid sequence encoding a BLUS1 polypeptide, wherein the one or more polymorphism(s) are associated with increased WUE.

In certain embodiments, the nucleic acid sequence is a genomic region encoding a BLUS1 polypeptide or promoter regulating expression of the BLUS1 polypeptide.

In certain embodiments, the method further comprises identifying one or more allele(s) at the one or more polymorphism(s) associated with increased WUE.

In certain embodiments, the invention provides a method of identifying (e.g. selecting) a plant having (or will have) reduced or abolished expression of at least one nucleic acid encoding a BLUS1 polypeptide and/or reduced or abolished activity of a BLUS1 polypeptide as described herein.

In certain embodiments, the invention provides a method of identifying a plant having increased WUE, increased leaf temperature, reduced gₛ under natural light, increased drought resistance and/or increased yield or biomass. For example, the one or more allele(s) may be used for marker-assisted selection of such plants.

Typically, the invention provides a method of detecting in the plant at least one marker that is indicative of reduced or abolished BLUS1 expression. For example, this marker may be a SNP or polymorphism in a nucleic acid sequence encoding a BLUS1 polypeptide as described herein. Alternatively, the marker may be a polymorphism at a highly associated marker locus, wherein the sequence at this locus is indicative of reduced or abolished BLUS1 expression (e.g. a linked marker). Typically, a linked marker is within about 10 cM, 5 cM, 4 cM, 3 cM, 2 cM, 1 cM or less from the causative mutation.

In certain embodiments, the at least one marker is selected from one or more of the following primers:

(a) to identify a mutant (e.g. Cadenza1445) having a substitution of C to T in a nucleic acid sequence corresponding to position 412 of SEQ ID NO: 3 (e.g. resulting in Q amino acid at position 138 of SEQ ID NO:4 mutated to a premature stop codon):

WT_F
(SEQ ID NO: 9)
CGCTCTGCTACCTTCACGAGc
Mut_F
(SEQ ID NO: 10)
CGCTCTGCTACCTTCACGAGt
Common_NB_R
(SEQ ID NO: 11)
GGAGTCGACGAGGACGTTc
(SEQ ID NO: 12)
(gAACGTCCTCGTCGACTCC);

(b) to identify a mutant (e.g. Cadenza0589) having a substitution of C to T in a nucleic acid sequence corresponding to position 1147 of SEQ ID NO: 3 (e.g. resulting in Q amino acid at position 383 of SEQ ID NO: 4 mutated to a premature stop codon):

WT_F1
(SEQ ID NO: 13)
GACAGCTTTGAGGATCTCGACc
MUT_F1
(SEQ ID NO: 14)
GACAGCTTTGAGGATCTCGACt
Common_A_specific_R1
(SEQ ID NO: 15)
CCGTGCTACCGTCTCAGTCt
(SEQ ID NO: 16)
(aGACTGAGACGGTAGCACGG);

(c) to identify a mutant (e.g. Cadenza0764) having a substitution of C to T in a nucleic acid sequence corresponding to position 1258 of SEQ ID NO: 5 (e.g. resulting in Q amino acid at position 420 of SEQ ID NO: 6 mutated to a premature stop codon):

WT_F
(SEQ ID NO: 17)
GATAAGGGAGTAACGGAAGAAc
Mut_F
(SEQ ID NO: 18)
GATAAGGGAGTAACGGAAGAAt
Common_B_specific_R2
(SEQ ID NO: 19)
CACCTCGAGGCTTAGATAGTCAc
(SEQ ID NO: 20)
(gTGACTATCTAAGCCTCGAGGTG);

(d) to identify a mutant (e.g. Cadenza0129) having a substitution of C to T in a nucleic acid sequence corresponding to position 497 of SEQ ID NO: 7 (e.g. resulting in S amino acid at position 420 of SEQ ID NO: 8 mutated to F):

WT_FAM
(SEQ ID NO: 21)
CCGACTTCGGCGTGTc
M_HEX
(SEQ ID NO: 22)
CCGACTTCGGCGTGTt
R_C
(SEQ ID NO: 23)
AGGAAGAAGAGCTGAGGACAAt
(SEQ ID NO: 24)
(aTTGTCCTCAGCTCTTCTTCCT);

(e) to identify a mutant (e.g. Cadenza1509) having a substitution of G to A in a nucleic acid sequence corresponding to position 770 of SEQ ID NO: 7 (e.g. resulting in R amino acid at position 257 of SEQ ID NO: 8 mutated to H):

WT_FAM
(SEQ ID NO: 25)
CTGATGAGGATCACGAGCCg
M_HEX
(SEQ ID NO: 26)
CTGATGAGGATCACGAGCCa
R_C
(SEQ ID NO: 27)
AGGCAGGAGGACACCATGt
(SEQ ID NO: 28)
(aCATGGTGTCCTCCTGCCT);

and/or

(f) to identify a mutant (e.g. J1-71) having a deletion across BLUS1 of wheat genome D as determined by primers for the BLUS1 gene (TraesCS5D02G203600) and an adjacent gene (TraesCS5D02G203100):

BLUS1_B_D_F
(SEQ ID NO: 29)
CGCTACGTAAGTATCATCACCAGT
BLUS1_D_ R1
(SEQ ID NO: 30)
CGAGGATGCCATTGTGTATCTGT
(SEQ ID NO: 31)
(ACAGATACACAATGGCATCCTCG)
_F
(SEQ ID NO: 32)
CACGTGCGCAGGAACATt
_R
(SEQ ID NO: 33)
GCCACGTGTTTCCATCCCt
(SEQ ID NO: 34)
(aGGGATGGAAACACGTGGC);

In certain embodiments, the invention provides a method for selecting plants having increased WUE by screening for the presence of any one or more marker(s) as described herein.

In certain embodiments, the invention provides use of any one or more marker(s) as described herein to select a plant having increased WUE. For example, the one or more marker(s) may comprise:
(i) any one or more of SEQ ID NOs: 9 to 34;
(ii) a nucleic acid sequence having at least about 85%, 90%, 95%, 98%, 99% or more identity with any one or more of SEQ ID NOs: 9 to 34;
(iii) a nucleic acid sequence having at least about 15 consecutive nucleotides of any one or more of SEQ ID NOs: 9 to 34; and/or
(iv) a marker within about 10 cM, 5 cM, 4 cM, 3 cM, 2 cM, 1 cM or less from the BLUS1 locus and indicative of reduced or abolished BLUS1 expression.

In certain embodiment, the plant is a polyploid. In such embodiments, the marker may be identified in one, two or more copies of the genome. For example, the plant may be hexaploid wheat. The marker may be identified in one, two or three copies of the genome.

Suitable tests for assessing the presence of a polymorphism are well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

In certain embodiments, the method may comprise obtaining a nucleic acid sample from the one or more plants. Such methods may further comprise carrying out amplification of the nucleic acid sequence encoding the BLUS1 polypeptide.

In certain embodiments, the nucleic acid sequence encoding the BLUS1 polypeptide is amplified by PCR. For example, one or more primers may be used to amplify the BLUS1 polypeptide such as those set forth in any one of SEQ ID NOs: 9 to 34.

In certain embodiments, the method may comprise introgressing the nucleic acid sequence comprising the one or more polymorphism(s) into a second plant. For example, the method may comprise introgressing the chromosomal region comprising the one or more polymorphism(s) into a different genetic background (e.g. an improved crop variety).

Typically, the one or more plants are crop plants (e.g. C₃ crop plants). For example, the one or more plants may be wheat (e.g. hexaploid wheat). In such embodiments, the second plant may be a drought and/or heat adapted background e.g. Sokoll, Berkut, Vorobey, Weebill, Cadenza, Reedling or Kingbird.

In certain embodiments, the wheat is hexaploid and comprises mutation(s) in the nucleic acid sequence encoding a BLUS1 polypeptide of:
(a) genome A and B but not D;
(b) genome A and D but not B,
(c) genome B and D but not A.

The mutations described in (a), (b) or (c) may reduce the expression of BLUS1 and/or activity of BLUS1. Typically, the mutations described in (a), (b) or (c) abolish the expression of BLUS1 and/or activity of BLUS1.

In certain embodiments, the wheat is hexaploid and comprises mutation(s) in the nucleic acid sequence encoding a BLUS1 polypeptide of genome A, B and D.

In certain embodiments, the mutations in genomes A and B abolish the expression of BLUS1 and the mutation(s) in genome D reduce the expression of BLUS1.

In certain embodiments, the mutations in genomes A and D abolish the expression of BLUS1 and the mutation(s) in genome B reduce the expression of BLUS1.

In certain embodiments, the mutations in genomes B and D abolish the expression of BLUS1 and the mutation(s) in genome A reduce the expression of BLUS1.

In certain embodiments, the mutations in genome A, B and D abolish the expression of BLUS1.

In certain embodiments, the one or more mutation(s) are identified by a TILLING method.

In certain embodiments, the invention provides a method of introducing and/or identifying mutation(s) in the nucleic acid sequence encoding a BLUS1 polypeptide of:
(a) genome A and B but not D;
(b) genome A and D but not B;
(c) genome B and D but not A; or
(d) genome A, B and D.

In certain embodiments, the one or more mutations in genome A are selected from:
a substitution of C to T in a nucleic acid sequence corresponding to position 412 of SEQ ID NO: 3;
a substitution in a nucleic acid sequence of SEQ ID NO:3 resulting in a substitution of Q to STP (e.g. Stop codon) in an amino acid sequence corresponding to position 138 of SEQ ID NO: 4;
a substitution of C to T in a nucleic acid sequence corresponding to position 1147 of SEQ ID NO: 3;
a substitution in a nucleic acid sequence of SEQ ID NO:3 resulting in a substitution of Q to STP (e.g. Stop codon) in an amino acid sequence corresponding to position 383 of SEQ ID NO: 4;
a substitution in a nucleic acid sequence of SEQ ID NO:3 resulting in a loss of BLUS-1 activity and/or loss of stomatal response to blue light; and/or
a substitution in a nucleic acid sequence of SEQ ID NO:3 resulting in a premature STOP codon.

In certain embodiments, the one or more mutations in genome B are selected from:
a substitution of C to T in a nucleic acid sequence corresponding to position 1258 of SEQ ID NO: 5;
a substitution in a nucleic acid sequence of SEQ ID NO:5 resulting in a substitution of Q to STP (e.g. Stop codon) in an amino acid sequence corresponding to position 420 of SEQ ID NO: 6;
a substitution in a nucleic acid sequence of SEQ ID NO:5 resulting in a loss of BLUS-1 activity and/or loss of stomatal response to blue light; and/or
a substitution in a nucleic acid sequence of SEQ ID NO:5 resulting in a premature STOP codon.

In certain embodiments, the one or more mutations in genome D are selected from:
a substitution of C to T in a nucleic acid sequence corresponding to position 497 of SEQ ID NO: 7;
a substitution in a nucleic acid sequence of SEQ ID NO:7 resulting in a substitution of S to F in an amino acid sequence corresponding to position 166 of SEQ ID NO: 8;
a substitution of G to A in a nucleic acid sequence corresponding to position 770 of SEQ ID NO: 7;
a substitution in a nucleic acid sequence of SEQ ID NO:7 resulting in a substitution of R to H in an amino acid sequence corresponding to position 257 of SEQ ID NO: 8;
a deletion across BLUS1-D1;
a substitution in a nucleic acid sequence of SEQ ID NO:7 resulting in a loss of BLUS-1 activity and/or loss of stomatal response to blue light; and/or
a substitution in a nucleic acid sequence of SEQ ID NO:7 resulting in a premature STOP codon.

In certain embodiments, the one or more mutations are identified using one or more primers selected from:
(a) one or more of SEQ ID NOs 9 to 12;
(b) one or more of SEQ ID NOs 13 to 16;
(c) one or more of SEQ ID NOs 17 to 20;
(d) one or more of SEQ ID NOs 21 to 24;
(e) one or more of SEQ ID NOs 25 to 28; and/or
(f) one or more of SEQ ID NOs 29 to 34.

In certain embodiments, the one or more BLUS1 mutations are introduced and/or backcrossed into a drought and/or heat adapted background of wheat e.g. Sokoll, Berkut, Vorobey or Weebill.

In certain embodiments, the one or more BLUS1 mutations are introduced and/or backcrossed into a Cadenza, Reedling or Kingbird background of wheat.

### High Throughput Screening of Plants

In certain embodiments, the invention provides a method of quantifying stomatal blue light response and/or photosynthetic capacity in one or more plants. Typically, the method comprises measuring stomatal conductance and/or leaf temperature in the one or more plants. The one or more plants (e.g. population of plants) may include any plant as described herein.

Typically, the invention provides a method of quantifying stomatal blue light response and/or photosynthetic capacity in one or more plants having reduced or abolished expression of at least one nucleic acid sequence encoding a BLUS1 polypeptide and/or reduced or abolished activity of a BLUS1 polypeptide. Typically, the one or more plants are wheat blus1 single, double and/or triple mutants as described herein.

In certain embodiments, the method comprises:
(a) acclimatizing the one or more plants to red light conditions;
(b) obtaining one or more measurements under the red light conditions;
(c) subjecting the one or more plants to blue light conditions; and
(d) obtaining one or more further measurements under the blue light conditions.

In certain embodiments, the red and/or blue light conditions comprise a photosynthetic photon flux density (PPFD) of between about 200 to about 800, between about 400 to about 600, typically about 500.

In certain embodiments, the red light conditions comprise 100% red light. Typically, the one or more plants are acclimatized to 100% red light (e.g. 0% blue light) prior to any measurement. For example, the one or more plants may be subjected to 100% red light for up to about one hour (e.g. for about 40 minutes) prior to any measurement.

In certain embodiments, the one or more measurements under the red light conditions are obtained over a further period of about 5 to about 15 minutes (e.g. about 10 minutes).

In certain embodiments, the one or more plants are subjected to blue light conditions following the first measurements under red light. For example, the plants may be subjected to at least about 5% blue light (e.g. plus about 95% red light), about 10% blue light (e.g. plus about 90% red light), about 15% blue light (e.g. plus about 85% red light), about 20% blue light (e.g. plus about 80% red light) about 25% blue light (e.g. plus about 75% red light), about 40% blue light (e.g. plus about 60% red light) or more. Typically, the plants are subjected to about 10% blue/about 90% red light.

In certain embodiments, the one or more measurements under blue light conditions are obtained over a period of about 20 to about 45 minutes. For example, the one or more measurements under blue light conditions may be obtained over a period of about 30 minutes.

Any suitable measurement may be taken to quantify the stomatal blue light response and/or photosynthetic capacity of the one or more plants.

In certain embodiments, leaf temperature is measured. Leaf temperature may be measured using any suitable techniques known in the art (e.g. using commercially available leaf temperature sensors).

In certain embodiments, stomatal conductance is measured. Stomatal conductance may be measured using any suitable techniques known in the art (e.g. using commercially available leaf poromoters or gas exchange chambers with infra-red analyzers).

Typically, the same technique is used for measuring leaf temperature and/or stomatal conductance under the red vs. blue light conditions. As such, the invention provides a method of quantifying a response of the one or more plants to a shift from red to blue light.

### Methods for Producing Food or Feed Products Under Water-Limited Conditions

In certain embodiments, the invention provides a method of increasing drought tolerance in a plant. For example, the method may comprise reducing or abolishing the expression of at least one nucleic acid encoding a BLUS1 polypeptide and/or reducing or abolishing the activity of a BLUS1 polypeptide according to any method described herein.

In certain embodiments, the method comprises producing a plant with reduced or abolished expression of at least one nucleic acid encoding a BLUS1 polypeptide and/or reduced or abolished activity of a BLUS1 polypeptide according to any method described herein.

In certain embodiments, the method comprises obtaining a plant part from the plant.

In certain embodiments, the method comprises producing a food or feed product from the plant part.

In certain embodiments, the invention provides use any plant or plant part as described herein to produce a food or feed product.

In certain embodiments, the food or feed product is non-propagation material such as flour, oil, fatty acids or the like.

In certain embodiments, the plant part is a seed.

In certain embodiments, the invention provides a seed produced from a genetically engineered or transgenic plant as described herein.

In the following, the invention will be explained in more detail by means of non-limiting examples of specific embodiments.

### Example 1—Removing the BL Response Reduces Stomatal Conductivity and Water Loss Whilst Maintaining Photosynthetic Carbon Gain

Traits related with photosynthetic capacity and efficiency are potential targets for improving yield as there is evidence that increasing photosynthesis will increase crop yields provided that other constraints do not become limiting. For optimal yield, it is important that plants optimise the use of the resources required for photosynthesis, in particular WUE.

Stomata control 99% of gaseous exchange between the external environment and the plant interior by opening and closing in response to environmental signals and internal cues, regulating CO₂ uptake for photosynthesis and water loss through transpiration. Water loss from the leaf is an order of magnitude greater than CO₂ uptake, due to differences in diffusion gradients, path length and the size of the molecules. Therefore, opening stomata for photosynthetic carbon fixation can compromise the water status of the plants. Stomatal behaviour is regulated by light, water status, CO₂ concentration, temperature and other environmental factors, as well as phytohormones such as abscisic acid that optimize plant growth in major environments.

In the field, light is one of the most dynamic environmental parameters that directly influences both photosynthesis and stomatal conductance. Stomata open in response to photosynthetically active radiation (PAR), which facilitates CO₂ diffusion into the leaf for photosynthesis, and often results in a close correlation between photosynthetic rates (A) and stomatal conductance (gₐ). However, gₛ is often greater than necessary for maximum photosynthesis under certain conditions resulting in unnecessary water loss with no carbon gain. While parameters such as photosynthetic rates and water-use efficiencies have been extensively studied or reviewed, the role of stomatal conductance to achieve higher yield potential has been largely neglected. Although stomatal behaviour controls photosynthesis and crop water use, it is currently an unexploited yet extremely promising target for manipulation to improve crop productivity.

Light regulation of stomata is mediated by the complex control of ion transport and solute biosynthesis in guard cells. Hyperpolarization of the plasma membrane by ATP driven proton pumps (H⁺-ATPase) results in the accumulation of ions and solutes, lowering the water potential and increasing water uptake, which causes guard cell swelling and pore opening. In C₃ crops such as wheat, two components of light-induced stomatal opening have been established and termed the ‘blue’ and the ‘red’ light response. The red light response, or photosynthesis-mediated response, saturates at similar light intensities as mesophyll photosynthesis and is thought to be mediated by intercellular CO₂ concentration (Cᵢ). The specific blue light response can be 20× more effective in opening stomata, even at intensities of blue light too low (˜5-15 μmol m⁻² s⁻¹) to drive photosynthesis and is enhanced by strong background red light.

As shown in FIG. 1, removing the BL response greatly reduces gₛ and water loss whilst maintaining photosynthetic carbon gain in wheat. Consequently, decreasing stomatal sensitivity to blue light optimises crop resource use, thereby maintaining photosynthetic rates while using water more efficiently. Decreasing water use enables sustained photosynthetic rates through the grain filling period when water becomes limiting, thus enhancing photosynthetic potential of the crop and overall grain yield.

When wheat was illuminated with PAR with the blue proportion of the spectrum removed (500-700 nm; see FIG. 1b), no differences in photosynthesis were apparent (FIG. 1c). However, gₛ values were significantly lower (30-40%) than when illuminated with the full PAR spectrum (400-700 nm; FIG. 1d). Intrinsic water use efficiency (water loss through stomata relative to CO₂ gain) was therefore greatly increased (ca. 52%) by the lower stomatal conductance when blue light was removed (FIG. 1e). Consequently, decreasing stomatal sensitivity to blue light within the PAR spectrum in wheat reduces stomatal aperture, greatly reducing unnecessary water loss without affecting photosynthetic rates. Reducing water use enables sustained photosynthetic rates throughout the crop cycle, thus enhancing overall photosynthetic potential and grain yield.

It is important that plants optimise the use of the water throughout the whole crop cycle without reducing CO₂ uptake to ensure sufficient water later in the season to maximize productivity and yield. Blue light in guard cells is perceived by membrane-associated receptor kinase phototropins (PHOT), PHOT1 and PHOT2. PHOTs are activated by auto-phosphorylation in the presence of blue light. Activated PHOTs each phosphorylate the Ser/Thr protein kinase, BLUS1 in a light intensity-dependent manner, leading to stomatal opening.

Arabidopsis blus1 mutants with missense mutations have shown the requirement of both the C and N terminus for proper BLUS activity, suggesting that both phosphorylation of the C-terminus and BLUS1 kinase activity are essential for BLUS1 signalling. Therefore, mutations in either the C or N terminus may abolish the BL signalling cascade. Importantly, this response may be restricted to guard cells as BLUS1 expression has been shown to be localized to stomatal guard cells and absent from the mesophyll.

As shown in FIG. 2, Arabidopsis blus1 mutants show reduced gₛ when 10 mmol m⁻² s⁻¹ of the total red light illumination (of 500 mmol m⁻² s⁻¹) was replaced with 10 mmol m⁻² s⁻¹ of blue, illustrating a decrease in gₛ due to reduction in perceived light intensity by these plants that are unable to process BL. Whilst in the WT plants gₛ increased by ca. 40% in response to the small increase in blue light, no change in photosynthetic rates was detected in either WT or mutant plants.

The magnitude of the blue light response is believed to be important in rapid early morning opening of stomata to facilitate high photosynthetic rates, however as illustrated in FIG. 1 removal of BL from the spectrum early in the morning did not impair photosynthesis. The blue light induced increase in gₛ also reduces leaf temperature by between 1-2° C. (FIG. 1) which could be important particularly in environments where warm temperatures limit photosynthesis.

### Example 2—Isolation of Blus1 Mutants in Wheat

Using publicly available TGAC gene models, a single triplet of genes homologous to Arabidopsis BLUS1 was identified on chromosome group 5 of wheat. The wheat and Arabidopsis BLUS1 polypeptides have over 65% identity across the kinase domain and have conservation of the phosphorylation motif in the C-terminus. The three wheat genes, TaBLUS1-A, B and D, are part of a distinct phylogenetic Glade which includes Arabidopsis BLUS1. These genes are expressed solely on leaves and on green tissues of spikes, but are not found expressed in any other wheat tissue examined, including multiple root samples, grain tissues, and non-photosynthetic organs such as ovary, stigmas, apical meristems, etc. Compared to other mesophyll expressed genes, TaBLUS1 has a relatively low expression level, most likely as a consequence of the mixture of cell types in wheat leaves. This expression pattern and level are consistent with a guard cell-specific expression pattern in wheat. The sequence homology, phylogenetic analysis and expression data suggest that these wheat genes are direct homologues of Arabidopsis BLUS1.

Loss of function Blus1 mutants in wheat were isolated by TILLING as follows:
seed is treated with ethyl methane sulfonate (EMS), sodium azide or gamma radiation. The treated seeds are sown and the resulting M1 plants grown to produce the next generation of seeds.
M1 plants are self-fertilized and the M2 seed harvested and sown. Each M2 plant is given a unique identifier. DNA is individually extracted from each M2 plant and stored in 96 well plates. The M3 seeds are harvested and catalogued for future sampling. The unique plant identifiers allow the DNA and the archived seeds to be cross-referenced and stored on a database.
to increase throughput, the M2 DNA samples are 8× pooled. Using gene-specific primers, PCR is carried out on the library of 8× pooled DNA samples. In the presence of a mutant, the amplification products when heated and cooled will form mismatched heteroduplexes between the wild type and mutant DNA.
to enable identification of the point mutations the re-annealed amplification products are incubated with a plant endonuclease called CELI, which preferentially cleaves at sites of heteroduplex mismatches that occur between wild-type and mutant DNA. The cleavage products are run on an automated fluorescence-based Capillary Electrophoresis System (e.g. Fragment Analyzer (Agilent)) that enables sensitive and high-resolution separation of DNA. Wells containing Cell digested fragments are identified as containing a mutant allele within the pool.
when a mutation is detected in the pooled DNA, PCR products amplified from the individual DNA samples that make up the pool are sequenced to identify the specific plant carrying the mutation. M3 seed from the mutant plant can then be accessed for sowing, genotyping and phenotypic analysis.

The exome-sequencing of 2,735 EMS-mutant lines of tetraploid (cv Kronos) and hexaploid (cv Cadenza) TILLING populations of wheat has allowed identification of non-sense and missense alleles for >90% of protein coding genes. This database was screened for mutations in the three TaBLUS1 gene models. From this screen, at least one truncation allele in each homolog resulting in a premature termination codon in the translated protein was identified providing a direct path to generate triple mutant TaBLUS1 null plants (FIG. 3).

The truncated mutations for all three homologs was confirmed. In order to track the mutation during crossing schemes, genome-specific KASP markers were developed as described herein. An additional set of missense mutations affecting many conserved sites within the kinase domain was also identified from the screen, as well as sites surrounding the phosphorylation motif in the C-terminus. This variation is expected to result in subtler effects on protein function compared to the knockout alleles.

TaBLUS1 homologs likely have overlapping functions given their similar protein sequence and expression patterns. Hence, triple null mutants with knock-out alleles for all three copies to account may be preferable. In some cases, dosage effects may be encountered where a triple null mutant might be too extreme from an agronomic perspective. In these cases, double mutants or combinations with missense alleles will be best suited to modulate the phenotype and improve quantitative traits such as yield. Dosage effects may be revealed through the generation of double null, triple null and combination of missense mutations in TaBLUS1 homologs.

Variation across the wheat TaBLUS1 homologs in the exome-sequenced hexaploid TILLING population has been identified. The isolated lines include mutations leading to premature termination codons, as well as more subtle single amino acid changes. This provides a non-transgenic route to delivering novel alleles into CIMMYT varieties to evaluate the effect of altered blue light response on yield across contrasting environments.

### Example 3—Generation of Double and Triple TaBLUS1 Mutants

The selected mutations are crossed into three CIMMYT varieties, including temperature tolerant wheat from the CIMMYT elite collection, and cultivars produced within existing IWYP programmes including increased grain size and enhanced photosynthetic rates (via SBPase). Double and triple TaBLUS1 knockout mutants are generated by combining the individual truncation alleles of the three Cadenza homologs using marker assisted selection.

To evaluate the possibility of TaBLUS1 dosage effects, double null mutants in the A and D genome are also combined with five different missense alleles from the B genome. The B genome is chosen as the donor of the five missense alleles given the position of the identified mutations within the kinase and phosphorylation motifs of the BLUS1 polypeptide. The double heterozygous A/D mutant (Aa/BB/Dd) is crossed with the five different B genome mutants (AA/bb/DD) and triple heterozygous individuals selected (Aa/Bb/Dd; 25% expected) and self-pollinated to select for triple mutants in the following generation. Based on the physiological results, the lines are further characterized in field trials.

These crossing schemes deliver Cadenza lines with one functional TaBLUS1 copy (double mutants), no functional TaBLUS1 (triple mutant), or a single mutant copy of TaBLUS1 with a predicted reduced functionality (double mutant+missense mutation). This allows dosage effect of TaBLUS1 to be evaluated and the best strategy to modulate the phenotype to maximize yield selected.

In parallel, the single knockout mutants are back-crossed into CIMMYT hexaploid varieties Reedling and Kingbird (part of IWYP76: grain size) and Sokoll (temperature tolerant synthetic background). Marker-assisted backcrossing is used to generate BC₃ single knockout lines which are combined into double and triple mutant combinations. The best performing mutant combinations are crossed into large grain size and enhanced photosynthetic rate lines.

### Example 4—Physiological Screening of Blus1 Mutants

Infra-red gas exchange analysis is used to physiologically quantify the effect on stomatal conductance (gₛ) and leaf temperature in selected mutants and crosses. In the different mutant backgrounds, photosynthetic performance and gₛ kinetics are determined under a range of environmental conditions relevant for yield potential. Thermal imaging is used as proxy for gₛ and chlorophyll fluorescence to determine photosynthetic efficiency.

The impact of blue light responses on double and triple mutants is assessed using thermography and gas exchange in a range of laboratory and field environments. Leaf water loss also determines leaf temperature. FIG. 1 shows that blue light-induced increases in gₛ reduced leaf temperature by ˜1-2° C. Therefore decreasing gₛ in certain environments may result in leaf temperatures that influence Rubisco activity and photosynthetic rates.

To produce germplasm for yield potential environments that could experience both temperature and water limitations to productivity, varieties are crossed that show tolerance to high temperature and water limitation associated with their synthetic backgrounds (Sokoll) with double and triple TaBLUS1 mutants. Any subtle variation in the triple mutants developed from double mutants combined with missense alleles may provide wheat with a modulated response for growth in increasingly variable agronomic environments. Thus, ideotypes with increased photosynthetic efficiency throughout the crop cycle due to optimum resource usage for diverse wheat mega environments are produced.

A dynamic thermal imaging system is used to assess gₛ under low and high irradiance (with and without a blue spectral component) to evaluate alterations in stomatal behaviour in the double, triple and missense mutants. The mutations cover the complete gene affecting conserved sites within the kinase domain as well as the 3′ phosphorylation site that could result in subtle variation in the individual phenotypes. Briefly, plants (mutants and WT controls) are placed under a spectrally controlled LED lighting system and gₛ stabilised at 1000 mmol m⁻² s⁻¹ red illumination to saturate photosynthesis. After 15 min, blue light illumination of 15 mmol m⁻² s⁻¹ is provided and BL responses of gₛ assessed through a decrease in temperature.

FIG. 4 provides an example of the thermography based screening described above, illustrating a decrease of 2° C. leaf temperature following 15 minutes of BL application, indicating a significant increase in gₛ and validating the screening approach. Illumination is achieved using a Valoya lighting system allowing full spectral control of illumination as well as intensity. Spatial images of gₛ are obtained from images of leaf temperature. The selected lines are subjected to detailed analysis using IRGAs and detailed physiological characterisation.

Photosynthetic efficiency is determined in all selected mutants/crosses using chlorophyll fluorescence and gas exchange to verify no adverse effects on photosynthetic capacity. The quantum efficiency of PSII photochemistry (F_{q}′/Fₘ′) establishes that light harvesting has been unaffected. Photosynthetic capacity of the flag leaf is determined using A/Cᵢ analysis [i.e. assimilation rate (A) as a function of intercellular CO₂ concentration (Cᵢ)]. The maximum carboxylation rates of Rubisco (Vcₘₐₓ), light and CO₂ saturated rate of photosynthesis (Amax) and the maximum rate of electron transport for RuBP regeneration (Jₘₐₓ) are determined.

Further detailed physiological analysis of gₛ and A responses are performed on selected lines to assess changes in stomatal behaviour and leaf temperature (See FIGS. 5 to 7). Steady-state characteristics are measured at a pre-defined light level (determined from A/Q), and kinetic responses in gₛ, A and Iwue recorded at intervals following step changes in irradiance (with and without a blue component). Leaf impressions determine if anatomical features have been altered.

To assess the impact of the mutation under different environmental conditions, physiological assessment of double and triple Cadenza and CIMMYT lines quantify the gₛ BL response and determine the impact of alterations in light, temperature and water status on physiology and biochemistry. Lines are grown in controlled environmental conditions and one key environmental parameter adjusted. Plants are subject to different watering regimes to emulate precipitation patterns such as those experienced in the field environment and subjected to the following measurements: leaf water and osmotic potential determined from leaf discs; photosynthetic measurements using chlorophyll fluorescence and gas exchange determined as described above. Stomatal kinetic responses of A, gₛ and ᵢWUE are assessed as described above; whilst diurnal gas exchange measurements determined under dynamic light regimes that mimic the field are used to assess integrated daily water use and photosynthetic rates. The putative associated effect of increased leaf temperature due to the mutations is evaluated in a second set of plants grown under the same conditions described above, with the exception that they are well-watered and growth temperature increased to 30° C. The same physiological and growth measurements described above is carried out. Additionally, the impact on Rubisco biochemistry and kinetics is determined. To assess the impact of growth light, lines are grown at high (2000 μmol m⁻² s⁻¹) or low-moderate irradiances (1000 μmol m⁻² s⁻¹) that represent Mexico and UK light regimes. The same physiological and growth measurements described above are carried out along with the impact on Rubisco activity assessed.

### Example 5—Physiological Characterisation of Selected Mutants in the Field

To assess the impact of the different allelic combinations of TaBLUS1 under field conditions, the mutants together with the parental lines are evaluated under two contrasting high yielding potential environments. These two environments are characterized for presenting high yields with contrasting evapotranspirative demand and different temperature during grain filling period. In both environments, detailed phenotypic characterization is carried out during years 2 and 3 in the double and triple null mutants and the triple missense mutants in the four different backgrounds (Cadenza, Reedling, Kingbird and Sokoll) under yield potential conditions. The trials include both the parental lines and the wild-type segregants from the mutant populations to account for background mutations when assessing yield and evaluate the possible trade-offs derived from the reduced stomatal conductance. The period of greater sensitivity of wheat crops appears to last between initiation of booting until a few days after anthesis, when grain number can be affected and lead to reduced grain yields. Grain number is established in the period between initiation of booting and seven to ten days after anthesis. This period is identified as rapid spike growth phase. Grain weight is determined during the grain filling period, from a few days after anthesis until physiological maturity. Growth analysis including in-season biomass at the key phenological stages (initiation of booting, GS41 and seven days after anthesis, GS65+7d) together with yield and yield components is evaluated. Photosynthetic potential is determined via leaf gas exchange measurements at the two key phenological stages, whilst diurnal and gₛ responses along the crop cycle is determined by thermal imaging using high resolution thermal cameras coupled in unmanaged aerial vehicles (UAVs). Together with gas exchange measurements, flag leaf samples are harvested in order to conduct biochemical analysis and characterise Rubisco activity. Thermography and biochemical assays determine the impacts on leaf temperature induced by the reduction in gₛ. Detailed growth and yield measurements assess overall performance under field conditions.

Samples (3-4 cm²) are collected from fully-illuminated leaves growing in controlled environment conditions, or in the field at JIC and CIMMYT, and rapidly frozen in liquid nitrogen, then stored at −80° C. until processing at LU. Protein leaf extracts are prepared from these samples and an aliquot quickly assayed for Rubisco activity. These data is used to calculate the activation state of Rubisco, which gives an indication of Rubisco activase function in the leaves. Separate aliquots from the same leaf extract is subsequently used to determine the amounts of: Rubisco (by ¹⁴CABP binding), Rubisco activase and any other photosynthetic proteins of interest (immunoblotting), total soluble protein (Bradford method) and chlorophyll (after extraction in ethanol).

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of increasing water use efficiency (WUE) in a plant, the method comprising reducing or abolishing the expression of at least one nucleic acid sequence encoding a Blue Light Signalling 1 (BLUS1) polypeptide and/or reducing or abolishing the activity of a BLUS1 polypeptide in the plant.

2. The method of claim 1, wherein WUE is increased by about 30% or more as compared to a control or wild-type plant.

3. The method of claim 1 or 2, wherein the method comprises introducing at least one mutation into the nucleic acid sequence encoding a BLUS1 polypeptide.

4. The method of claim 3, wherein the mutation is a loss of function mutation, optionally an insertion, deletion or substitution.

5. The method of claim 3 or 4, wherein the mutation is introduced by insertional mutagenesis, optionally transposon mutagenesis.

6. The method of claim 3 or 4, wherein the mutation is identified by a Targeted Induced Local Lesions in Genomics (TILLING) method.

7. The method of claim 3 or 4, wherein the mutation is introduced by genome-editing, optionally CRISPR/Cas9.

8. The method of claim 1 or 2, wherein a transgenic construct is introduced into the plant.

9. The method of claim 8, wherein the transgenic construct is capable of reducing or abolishing the expression of the nucleic acid encoding a BLUS1 polypeptide by RNA interference, sense suppression, antisense suppression and/or miRNA suppression.

10. The method of any one of the preceding claims, wherein the nucleic acid sequence encoding the BLUS1 polypeptide comprises SEQ ID NO: 1, 3, 5 or 7 or a sequence having at least 80% homology to SEQ ID NO:1, 3, 5 or 7 based on nucleic acid identity over the entire length of the sequence.

11. The method of any one of the preceding claims, wherein the plant is a crop plant, optionally wheat.

12. The method of claim 11, wherein the plant is hexaploid wheat and the method comprises introducing and/or identifying mutation(s) in the nucleic acid sequence encoding a BLUS1 polypeptide of:
(a) genome A and B but not D;
(b) genome A and D but not B;
(c) genome B and D but not A; or
(d) genome A, B and D.

13. A plant, plant part or seed obtainable by the method of any one of claims 1 to 12.

14. A plant having reduced or abolished expression of at least one nucleic acid encoding a BLUS1 polypeptide and/or reduced or abolished activity of a BLUS1 polypeptide.

15. The plant of claim 14, wherein the plant is a crop plant, optionally wheat.

16. The plant of claim 14 or 15, wherein the plant is temperature tolerant.

17. The plant of any one of claims 14 to 16, wherein the plant has increased WUE relative to a control or wild-type plant.

18. The plant of claim 17, wherein WUE is increased by about 30% or more as compared to a control or wild-type plant a control or wild-type plant.

19. The plant of any one of claims 14 to 18, wherein the plant comprises one or more mutations in at least one nucleic acid sequence encoding a BLUS1 polypeptide.

20. The plant of claim 19, wherein:
(a) the mutation is introduced by insertional mutagenesis, optionally transposon mutagenesis;
(b) the mutation is identified by a TILLING method; or
(c) the mutation is introduced by genome-editing, optionally CRISPR/Cas9.

21. The plant of any one of claims 14 to 17, wherein the plant comprises a transgenic construct.

22. The plant of claim 21, wherein the transgenic construct is capable of reducing or abolishing the expression of the nucleic acid encoding a BLUS1 polypeptide by RNA interference, sense suppression, antisense suppression and/or miRNA suppression.

23. The plant of any one of claims 14 to 22, wherein the plant is hexaploid wheat and comprises mutation(s) in the nucleic acid sequence encoding a BLUS1 polypeptide of:
(a) genome A and B but not D;
(b) genome A and D but not B; or
(c) genome B and D but not A;
optionally wherein the mutation(s) in each genome abolish the expression of BLUS1.

24. The plant of any one of claims 14 to 22, wherein the plant is hexaploid wheat and comprises mutation(s) in the nucleic acid sequence encoding a BLUS1 polypeptide of genome A, B and D, optionally wherein:
(a) the mutation(s) in genomes A and B abolish the expression of BLUS1 and the mutation(s) in genome D reduce the expression of BLUS1;
(b) the mutation(s) in genomes A and D abolish the expression of BLUS1 and the mutation(s) in genome B reduce the expression of BLUS1;
(c) the mutation(s) in genomes B and D abolish the expression of BLUS1 and the mutation(s) in genome A reduce the expression of BLUS1; or
(d) the mutation(s) in genomes A, B and D abolish the expression of BLUS1.

25. The method of claim 12 or plant of claim 23 or 24, wherein:
(a) the mutation(s) in genome A are selected from:
(i) a substitution of C to T in a nucleic acid sequence corresponding to position 412 of SEQ ID NO: 3;
(ii) a substitution in a nucleic acid sequence of SEQ ID NO:3 resulting in a substitution of Q to STP (e.g. Stop codon) in an amino acid sequence corresponding to position 138 of SEQ ID NO: 4;
(iii) a substitution of C to T in a nucleic acid sequence corresponding to position 1147 of SEQ ID NO: 3; and/or
(iv) a substitution in a nucleic acid sequence of SEQ ID NO:3 resulting in a substitution of Q to STP (e.g. Stop codon) in an amino acid sequence corresponding to position 383 of SEQ ID NO: 4;
(b) the mutation(s) in genome B are selected from:
(i) a substitution of C to T in a nucleic acid sequence corresponding to position 1258 of SEQ ID NO: 5; and/or
(ii) a substitution in a nucleic acid sequence of SEQ ID NO: 5 resulting in a substitution of Q to STP (e.g. Stop codon) in an amino acid sequence corresponding to position 420 of SEQ ID NO: 6; and/or
(c) the mutation(s) in genome D are selected from:
(i) a substitution of C to T in a nucleic acid sequence corresponding to position 497 of SEQ ID NO: 7;
(ii) a substitution in a nucleic acid sequence of SEQ ID NO: 7 resulting in substitution of S to F in an amino acid sequence corresponding to position 166 of SEQ ID NO: 8;
(iii) a substitution of G to A in a nucleic acid sequence corresponding to position 770 of SEQ ID NO: 7;
(iv) a substitution in a nucleic acid sequence of SEQ ID NO: 8 resulting in a substitution of R to H in an amino acid sequence corresponding to position 257 of SEQ ID NO: 8; and/or
(v) a deletion across BLUS1-D1; and/or
(d) the one or more mutation(s) are selected from:
(i) a substitution in a nucleic acid sequence of SEQ ID NO: 3, 5 and/or 7 resulting in a loss of BLUS-1 activity and/or loss of stomatal response to blue light; and/or
(ii) a substitution in a nucleic acid sequence resulting in a premature STOP codon.

26. A plant part or seed obtained from the plant of any one of claims 14 to 25, optionally wherein the seed comprises the mutation as defined in claim 19 or 20 or the transgenic construct as defined in claim 21 or 22.

27. A method of identifying one or more alleles associated with increased WUE in one or more plants, the method comprising:
(a) detecting in the plant(s) one or more polymorphism(s) in a nucleic acid sequence encoding a BLUS1 polypeptide, wherein the one or more polymorphism(s) are associated with increased WUE;
(b) identifying one or more allele(s) at the one or more polymorphism(s) that are associated with increased WUE.

28. The method of claim 27, wherein the one or more polymorphism(s) are identified using one or more primer(s) selected from:
(a) one or more of SEQ ID NOs 9 to 12;
(b) one or more of SEQ ID NOs 13 to 16;
(c) one or more of SEQ ID NOs 17 to 20;
(d) one or more of SEQ ID NOs 21 to 24;
(e) one or more of SEQ ID NOs 25 to 28; and/or
(f) one or more of SEQ ID NOs 29 to 34.

29. The method of claim 28, further comprising introgressing the nucleic acid sequence comprising the one or more polymorphism(s) into a temperature tolerant plant.

30. The method of claim 28 or 29, wherein the plant is a crop plant, optionally wheat.

31. A method of producing a food or feed product under water-limited or drought conditions, the method comprising:
(a) obtaining a plant with reduced or abolished expression of at least one nucleic acid encoding a BLUS1 polypeptide and/or reduced or abolished activity of a BLUS1 polypeptide according to the method of any one of claims 1 to 12 or plant of any one of claims 14 to 25;
(b) isolating a plant part or seed from the plant; and
(c) producing a food or feed product from the plant part or seed.

32. A method of quantifying stomatal blue light response and/or photosynthetic capacity in one or more plants having reduced or abolished expression of at least one nucleic acid sequence encoding a BLUS1 polypeptide and/or reduced or abolished activity of a BLUS1 polypeptide, wherein the method comprises:
(a) acclimatizing the one or more plants to red light conditions;
(b) obtaining one or more measurements under the red light conditions;
(c) subjecting the one or more plants to blue light conditions; and
(d) obtaining one or more further measurements under the blue light conditions.

33. The method of claim 32, wherein stomatal conductance and/or leaf temperature is measured.

34. Use of a plant of any one of claims 15 to 26 for producing a food or feed product.

35. Use according to claim 34, wherein:
(a) the plant is a crop plant, optionally wheat; and/or
(b) the food or feed product is a non-propagation material such as flour or oil.


SEQUENCE LISTING (34 sequences)

SEQ ID NO: 1 - DNA, 1959 bp, Arabidopsis thaliana
gtttgaagttgaaatgatagtgtttgccctttgggtccctggaaagaccaaaggtaaaatctcattaactaaggttagttagttagctctttcacatgcttcatcttattgtttcatcaacatatttttaagtaagagataagggttttgcttttacattaaaggctctcttttttttcaagttcacatgctaaaccaactctttcttttttcatccctatatgaatagcctcatggtactattagcctctaaaagaccacaattccactaaaaacataacaattcaataagcaagagtgtactcatcttctttctatttatggctcggaacaagctcgagttccctcttgatgctgaagcctacgagatcatctgcaagataggcgttggtgttagtgcttcggtctacaaggccatatgcattccgatgaactcaatggtagttgctatcaaagccatcgatcttgatcagtcgcgggctgactttgacagtcttcgccgtgaaaccaagacgatgtctctgctttctcatccgaatattctcaatgcttattgttcattcaccgttgatcgatgtctctgggtggttatgccattcatgtcttgtggctctcttcattcgatcgtctcttcgtcttttccaagtgggttaccagaaaactgcatttccgtcttcctcaaggaaactctgaatgcaatctcgtatcttcacgatcagggtcatttgcaccgtgacatcaaggcaggtaacattctggtagattctgatggatccgtgaagctcgctgatttcggagtatctgcatccatctatgaacccgtgacatcttcctctggaacaacatcttcttctttaaggttaactgatatagcgggaacaccgtattggatggctccggaagtggttcattcccacacagggtatggtttcaaagcagacatttggtctttcgggataacagcgttggagttagcacatggaagacctccgttatctcacttaccgccgttgaagagtctgctcatgaagatcaccaaaaggtttcatttttctgattacgagatcaatacgagcggaagcagcaaaaagggtaacaagaagttctcaaaagcttttagagaaatggttggtttgtgtctagagcaagatcctactaaaagaccatcggcagagaagttgttgaagcatccttttttcaagaactgtaaaggactcgactttgtggtcaagaacgtgttgcatagcttgtcaaacgcagagcagatgtttatggagagtcagattttgatcaagagtgttggagatgatgatgaagaagaagaagaagaagacgaagagatagtgaagaatagaagaatcagtgggtggaatttccgtgaagacgatctccaacttagtccagtgttcccagctactgaatcagactcttctgagtccagtccacgtgaagaagatcaatcaaaagacaaaaaggaagacgataacgtcacaataacggggtatgaactcggtttaggtttgtcgaacgaggaagctaagaaccaagaaggtgaggttgttgggtttgataaagatttggtgttagagaaactgaaagtgttgaagaaaagtttagagcatcaaagagcaagagtgtcgattataatcgaagcattgagtggggacaaggaagagaagagcagagaagaagagcttctagagatggtggagaagttaaagattgaattggaaactgagaagctaaagaccttgcgtgctgataaagatagtgttttgggttaactattctaaacttgttaatattttttttctatatgctaaaattatataagtggcatgacattacgatcaattgttttcaatttaactcgttttatttgtcagtttagaatggctccaaattaagttttggtaaacaagttatatattcaccaaaacaacatataaatatttcctc

SEQ ID NO: 2 - protein, 487 aa, Arabidopsis thaliana
MARNKLEFPLDAEAYEIICKIGVGVSASVYKAICIPMNSMVVAIKAIDLDQSRADFDSLRRETKTMSLLSHPNILNAYCSFTVDRCLWVVMPFMSCGSLHSIVSSSFPSGLPENCISVFLKETLNAISYLHDQGHLHRDIKAGNILVDSDGSVKLADFGVSASIYEPVTSSSGTTSSSLRLTDIAGTPYWMAPEVVHSHTGYGFKADIWSFGITALELAHGRPPLSHLPPLKSLLMKITKRFHFSDYEINTSGSSKKGNKKFSKAFREMVGLCLEQDPTKRPSAEKLLKHPFFKNCKGLDFVVKNVLHSLSNAEQMFMESQILIKSVGDDDEEEEEEDEEIVKNRRISGWNFREDDLQLSPVFPATESDSSESSPREEDQSKDKKEDDNVTITGYELGLGLSNEEAKNQEGEVVGFDKDLVLEKLKVLKKSLEHQRARVSIIIEALSGDKEEKSREEELLEMVEKLKIELETEKLKTLRADKDSVLG

SEQ ID NO: 3 - DNA, 1557 bp, Triticum aestivum
atggcagacgacgcggggcctggcggcgaggccaagtacccgctcaacccggattgctaccgcctgctctgcaagatcgggagcggcgtcagcgccgtcgtgtacaaggccgcgtgcctgccgctcggctccgtgccggtggccatcaaggccatcgacctcgagcgctcccgcgccaacctggaggacgtgtggcgggaggccaaggccatggcgctcctgtcgcacgccaacgtcctgcgcgcgcactgctccttcaccgtgggcagccacctgtgggtggtcatgccgttcatggccgccggctctctgcactccatcctggcccacggcttccccgacggcctcccggagccgtgcatcgcggtggtgctcaaggagacgctccgtgcgctctgctaccttcacgagcagggccgcatccaccgcgacatcaaggccgggaacgtcctcgtcgactccgacggctccgtcaagctcgccgacttcggcgtgtccgcgtccatatacgagaccccgccgccggcgtcgtccttctccgggcccctgacccacgccccccaggttgtcctcagctcttcttcctacttcagcgagatggcagggacgccgtactggatggcgccggaggtcatccactcgcacgtcggctacggcatcaaggccgacatctggtcgttcggcatcacggccctggagctcgcgcacggccggccgcccctctcccacctgccgccgtccaagtcgatgctgatgaggatcacgagccgcgtccggatggaggacgcggagatctccaagaacaagaagctatccaaggcgttcaaggacatggtgtcctcctgcctgtgccaagagccggccaagcggccgtcggcggagaagcttctccggcacccgttcttcaaaggctgccgctccaaggactacctcgtccgcaacgtcctcagcgtcgtgccgagcatcgaggagcgctgcaaggacgtcacgggcctctgcggctgcgccgccgggggcgcgcgctgcgtgtcgccgtgccacggccaggcgagcgcgagcatcgtcaagaaccgccgcatgagcggctggaacttcggcgcggactgcccgaggaaggaagacgcggacagctttgaggatctcgaccagactgagacggtagcacggctgttccttccactcgacgacgaggacacggtgcccgagcgggcctgcgacggggccggagaagatggagacaagggaacaatggagcagcaaggtgatcgagaggaaaacgagggatcgttcggcgtgaaaggggtggtggtgccgcatctgatgactatcttgggaagcctcgaggtgcagaagaggatgctggcccaagaactggaaggtggctgctgctatcaccacgacggcaactgctgccgcgagacgacggccagggaggagatgctactcgcgtacgtgcgtcagctcgagcagagggtggaggtgctgaccttggaggtcgaggaggaaatcgccaggaatgcccacctggaggagcttcttcgtgagagggctggttga

SEQ ID NO: 4 - protein, 518 aa, Triticum aestivum
MADDAGPGGEAKYPLNPDCYRLLCKIGSGVSAVVYKAACLPLGSVPVAIKAIDLERSRANLEDVWREAKAMALLSHANVLRAHCSFTVGSHLWVVMPFMAAGSLHSILAHGFPDGLPEPCIAVVLKETLRALCYLHEQGRIHRDIKAGNVLVDSDGSVKLADFGVSASIYETPPPASSFSGPLTHAPQVVLSSSSYFSEMAGTPYWMAPEVIHSHVGYGIKADIWSFGITALELAHGRPPLSHLPPSKSMLMRITSRVRMEDAEISKNKKLSKAFKDMVSSCLCQEPAKRPSAEKLLRHPFFKGCRSKDYLVRNVLSVVPSIEERCKDVTGLCGCAAGGARCVSPCHGQASASIVKNRRMSGWNFGADCPRKEDADSFEDLDQTETVARLFLPLDDEDTVPERACDGAGEDGDKGTMEQQGDREENEGSFGVKGVVVPHLMTILGSLEVQKRMLAQELEGGCCYHHDGNCCRETTAREEMLLAYVRQLEQRVEVLTLEVEEEIARNAHLEELLRERAG

SEQ ID NO: 5 - DNA, 1557 bp, Triticum aestivum
atggcagacgaggcgggggctggcggcgaagccaagtacccgctcaacccagagtgctaccgcctgctctgcaagatcgggagcggcgtcagcgccgtcgtgtacaaggccgcgtgcctgccgctcggctcggtgccggtggccatcaaggccatcgacctcgagcgttcccgcgccaacctggaggacgtgtggcgggaggccaaggccatggcgctcctgtcgcacgccaacgtcctgcgcgcgcactgctccttcaccgtgggcagccacctgtgggtggtcatgccgttcatggccgccggctcgctgcactccatcctggcccatggcttccccgacggcctcccggagccgtgcatcgcggtggtactcaaggagacgctccgtgcgctctgctaccttcacgagcagggccgcatccaccgcgacatcaaggccgggaacctcctcgtcgactctgacggctccgtcaagctcgccgacttcggcgtgtccgcgtccatatacgagaccccgccgccggcgtcgtccttctccgggcccttgacacacgcccctcaggttgttctcagctcttcttcctacttcagcgagatggcagggacgccgtactggatggcgccggaggtcatccactcgcacgtcggctacggcatcaaggccgacatctggtcgttcggcatcacggccctcgagctcgcgcacggccggccgcccctctcccacctgccgccgtccaagtcgatgctgatgaggatcacgagccgcgtccggatggaggacgcggagatctccaagaacaagaagctctccaaggcgttcaaggccatggtgtcctcctgcctgtgccaagagccggccaagcggccatcggcggagaagcttctccggcacccgttcttcaaaggctgccgctccaaggactacctagtgcgcaacgtcctcagcatcgtgccgagcatcgaggagcgctgcaaggacgtcacgggcctctgcggctgcgccgccgggggcgcgcgctgcgtgtcgccgtgccacggccaggcgagcgcgagcatcgtcaagaaccgccgaatgagcggctggaacttcggcgcggactgcccgagaaaggaagacgcggacagctttgaggagctcgaccggaccgagacggtagcacggctattccttccactcgacgacgaggacaccgtgcccgagcgggcctgcgaaggcgccggagaagacggagataagggagtaacggaagaacaatgtgatcgagaagaaaacgacggatcgtttggcgtgaaaggggtggtggtgccgcatctggtgactatcttgggaagcctcgaggtgcagaaacggatgctggcccaagaactggaaggtgggtgctgctatcaccacaacggcaactgctgccgcgagacgacggccagggaggagatgctactcgcgtacgtgcgtcagctcgagcagagggtggaggtgctgaccctggaggtagaggaggaaatcaccaggaatgcccacctggaggagcttcttcgtgggagggctggttga

SEQ ID NO: 6 - protein, 518 aa, Triticum aestivum
MADEAGAGGEAKYPLNPECYRLLCKIGSGVSAVVYKAACLPLGSVPVAIKAIDLERSRANLEDVWREAKAMALLSHANVLRAHCSFTVGSHLWVVMPFMAAGSLHSILAHGFPDGLPEPCIAVVLKETLRALCYLHEQGRIHRDIKAGNLLVDSDGSVKLADFGVSASIYETPPPASSFSGPLTHAPQVVLSSSSYFSEMAGTPYWMAPEVIHSHVGYGIKADIWSFGITALELAHGRPPLSHLPPSKSMLMRITSRVRMEDAEISKNKKLSKAFKAMVSSCLCQEPAKRPSAEKLLRHPFFKGCRSKDYLVRNVLSIVPSIEERCKDVTGLCGCAAGGARCVSPCHGQASASIVKNRRMSGWNFGADCPRKEDADSFEELDRTETVARLFLPLDDEDTVPERACEGAGEDGDKGVTEEQCDREENDGSFGVKGVVVPHLVTILGSLEVQKRMLAQELEGGCCYHHNGNCCRETTAREEMLLAYVRQLEQRVEVLTLEVEEEITRNAHLEELLRGRAG

SEQ ID NO: 7 - DNA, 1557 bp, Triticum aestivum
atggcagacgacgcgggggccggcggcgaagccaaatacccgctcaacccagagtgctaccgcctgctctgcaagatcggaagcggcgtcagcgccgtcgtgtacaaggccgcgtgcctgccgctcggttccgtgccggtggccatcaaggccatcgacctcgagcgctcccgcgccaacctggaggacgtgtggcgggaggccaaggccatggcgctcctgtcgcacgccaacgtcctgcgcgcgcactgctccttcacggtgggcagccacctatgggtggtcatgccgttcatggccgccggctcgctgcactccatcctcgcccacggcttccccgacggcctaccagagccgtgcatcgcggtggtgctcaaggagacgctccgtgcgctctgctaccttcacgagcagggccgcatccaccgcgacatcaaggccggaaacgtcctcgtcgactccgacggctccgtcaagctcgccgacttcggcgtgtccgcgtccatatacgagaccccgccgccggcgtcgtccttctccgggcccttgacacacgcccctcagattgtcctcagctcttcttcctacttcagcgagatggcagggacgccgtactggatggcgccggaggtcatccactcgcacgtcggctacggcatcaaggccgacatctggtcgttcggcatcacggccctggagctcgcgcacggccggccgcccctctcccacctgccgccgtccaagtcgatgctgatgaggatcacgagccgcgtccggatggaggacgcggagatctccaagaacaagaagctctccaaggcgttcaaggacatggtgtcctcctgcctgtgccaagagccggccaagcggccatcggcggagaagcttctccggcatccgttcttcaaaggctgccgctccaaggaccacctcgtccgcaacgtcctcagcgtcgtgccgagcatcgaggagcgctgcaaggacgtcacgggcctctgcggctgcgccgccgggggcgcgcgctgcgtgtcgccgtgccacggccaggcgagcgcgagcatcgtcaagaaccgccgcatgagcggctggaacttcggcgcggactgcccgaggaaggaagacgcggacagctttgaggagctcgaccggaccgagacgacagcacggctgttccttccactcgacgacgaggacaccgtgcctgagcggacctgcgaaggcgccggagaagacggagataagggagtaacggaagaacaaggtgatcgagaagaaaacgagggatcgtttggcgtgaaaggggtggtggtgccgcatctgatgactatcttgggaagcctcgaggtgcagaaacggatgctggcccaagaactggaaggtggctgctgctatcaccacgacggcaactgctgcggcgagacgacggccagggaggagatgctactcgcgtacgtacgtcagctcgagcagagggtggagatgctgaccttggaggtagaggaggaaatcaccaggaatgcccagctggaggagcttcttcgtgagagggctggttaa

SEQ ID NO: 8 - protein, 518 aa, Triticum aestivum
MADDAGAGGEAKYPLNPECYRLLCKIGSGVSAVVYKAACLPLGSVPVAIKAIDLERSRANLEDVWREAKAMALLSHANVLRAHCSFTVGSHLWVVMPFMAAGSLHSILAHGFPDGLPEPCIAVVLKETLRALCYLHEQGRIHRDIKAGNVLVDSDGSVKLADFGVSASIYETPPPASSFSGPLTHAPQIVLSSSSYFSEMAGTPYWMAPEVIHSHVGYGIKADIWSFGITALELAHGRPPLSHLPPSKSMLMRITSRVRMEDAEISKNKKLSKAFKDMVSSCLCQEPAKRPSAEKLLRHPFFKGCRSKDHLVRNVLSVVPSIEERCKDVTGLCGCAAGGARCVSPCHGQASASIVKNRRMSGWNFGADCPRKEDADSFEELDRTETTARLFLPLDDEDTVPERTCEGAGEDGDKGVTEEQGDREENEGSFGVKGVVVPHLMTILGSLEVQKRMLAQELEGGCCYHHDGNCCGETTAREEMLLAYVRQLEQRVEMLTLEVEEEITRNAQLEELLRERAG

SEQ ID NO: 9 - DNA, 21 bp, Artificial Sequence
  WT_F
cgctctgctaccttcacgagc

SEQ ID NO: 10 - DNA, 21 bp, Artificial Sequence
  Mut_F
cgctctgctaccttcacgagt

SEQ ID NO: 11 - DNA, 19 bp, Artificial Sequence
  Common_A/B_R
ggagtcgacgaggacgttc

SEQ ID NO: 12 - DNA, 19 bp, Artificial Sequence
  Common_A/B_R
gaacgtcctcgtcgactcc

SEQ ID NO: 13 - DNA, 22 bp, Artificial Sequence
  WT_F1
gacagctttgaggatctcgacc

SEQ ID NO: 14 - DNA, 22 bp, Artificial Sequence
  MUT_F1
gacagctttgaggatctcgact

SEQ ID NO: 15 - DNA, 20 bp, Artificial Sequence
  Common_A_specific_R1
ccgtgctaccgtctcagtct

SEQ ID NO: 16 - DNA, 20 bp, Artificial Sequence
  Common_A_specific_R1
agactgagacggtagcacgg

SEQ ID NO: 17 - DNA, 22 bp, Artificial Sequence
  WT_F
gataagggagtaacggaagaac

SEQ ID NO: 18 - DNA, 22 bp, Artificial Sequence
  Mut_F
gataagggagtaacggaagaat

SEQ ID NO: 19 - DNA, 23 bp, Artificial Sequence
  Common_B_specific_R2
cacctcgaggcttagatagtcac

SEQ ID NO: 20 - DNA, 23 bp, Artificial Sequence
  Common_B_specific_R2
gtgactatctaagcctcgaggtg

SEQ ID NO: 21 - DNA, 16 bp, Artificial Sequence
  WT_FAM
ccgacttcggcgtgtc

SEQ ID NO: 22 - DNA, 16 bp, Artificial Sequence
  M_HEX
ccgacttcggcgtgtt

SEQ ID NO: 23 - DNA, 22 bp, Artificial Sequence
  R_C
aggaagaagagctgaggacaat

SEQ ID NO: 24 - DNA, 22 bp, Artificial Sequence
  R_C
attgtcctcagctcttcttcct

SEQ ID NO: 25 - DNA, 20 bp, Artificial Sequence
  WT_FAM
ctgatgaggatcacgagccg

SEQ ID NO: 26 - DNA, 20 bp, Artificial Sequence
  M_HEX
ctgatgaggatcacgagcca

SEQ ID NO: 27 - DNA, 19 bp, Artificial Sequence
  R_C
aggcaggaggacaccatgt

SEQ ID NO: 28 - DNA, 19 bp, Artificial Sequence
  R_C
acatggtgtcctcctgcct

SEQ ID NO: 29 - DNA, 24 bp, Artificial Sequence
  BLUS1_B_D_F
cgctacgtaagtatcatcaccagt

SEQ ID NO: 30 - DNA, 23 bp, Artificial Sequence
  BLUS1_D_ R1
cgaggatgccattgtgtatctgt

SEQ ID NO: 31 - DNA, 23 bp, Artificial Sequence
  BLUS1_D_ R1
acagatacacaatggcatcctcg

SEQ ID NO: 32 - DNA, 18 bp, Artificial Sequence
  _F
cacgtgcgcaggaacatt

SEQ ID NO: 33 - DNA, 19 bp, Artificial Sequence
  _R
gccacgtgtttccatccct

SEQ ID NO: 34 - DNA, 19 bp, Artificial Sequence
  _R
agggatggaaacacgtggc